(19) United States
(12) Patent Application Publication
(10) Pub. No.: US 2017/0304197 A1
(43) Pub. Date: Oct. 26, 2017

(54) FORMULATIONS CONTAINING TIOTROPIUM, AMINO ACID AND ACID AND METHODS THEREOF

(71) Applicant: PULMATRIX OPERATING COMPANY, INC., Lexington, MA (US)

(72) Inventors: Jean C. Sung, Cambridge, MA (US); Jason M. Perry, Cambridge, MA (US); Michael Tauber, Allston, MA (US)

(73) Assignee: PULMATRIX OPERATING COMPANY, INC., Lexington, MA (US)

(21) Appl. No.: 15/517,724

(22) PCT Filed: Oct. 7, 2015
(86) PCT No.: PCT/US15/54447

§ 371 Date: Apr. 7, 2017

Related U.S. Application Data
(60) Provisional application No. 62/061,436, filed on Oct. 8, 2014.

(51) Int. Cl. A61K 9/00; C07D 333/10; A61K 31/58; A61K 31/46; A61K 31/439; C07D 419/14; A61K 9/14

(52) U.S. Cl. CPC A61K 9/0075; C07D 419/14; C07D 333/10; A61K 9/145; A61K 31/46; A61K 31/439; A61K 31/58

(57) ABSTRACT

A dry powder containing dry particles that contain a tiotropium salt, one or more amino acids, and acid content, and optionally, sodium chloride, and/or one or more additional therapeutic agents, wherein the molar ratio of acid to amino acid is from about 0.0005 to about 5, or 0.002 to about 1. In one aspect, the dry powder containing dry particles is suitable for administration to the respiratory tract. In one aspect, the dry powder containing dry particles is a respirable dry powder contains respirable dry particles that contain a tiotropium salt, one or more amino acids, acid content, sodium chloride, and optionally one or ore additional therapeutic agents, wherein the tiotropium salt is about 0.01% to about 0.5%, the leucine is about 5% to about 40%, the sodium chloride is about 50% to about 90%, the optional one or more additional therapeutic agents are up to about 30%, and the molar ratio of acid to amino acid is from about 0.002 to about 1, where all percentages are weight percentages on a dry basis and all the components of the respirable dry particles amount to 100%.

### BACKGROUND

The chemical structure of tiotropium was first described in U.S. Pat. No. 5,610,163 and RE39,820. Tiotropium salts include salts containing cationic tiotropium with one of the following anions: bromide, fluoride, chloride, iodine, C1-C4-alkylsulphate, sulphate, hydrogen sulphate, phosphate, hydrogen phosphate, di-hydrogen phosphate, nitrate, maleate, acetate, trifluoroacetate, citrate, fumarate, tartrate, oxalate, succinate and benzoate, C1-C4-alkylsulphonate, which may optionally be mono-, di- or tri-substituted by fluorine at the alkyl group, or phenylsulphonate, which may optionally be mono- or poly-substituted by C1-C4-alkyl at the phenyl ring. Tiotropium bromide is an anticholinergic providing therapeutic benefits, e.g. in the treatment of COPD and asthma, and is the active ingredient in SPIRIVA (tiotropium bromide) HANDIHALER (dry powder inhaler) (Boehringer Ingelheim, Germany). Tiotropium bromide is known to crystallize in various forms, such as crystalline anhydrous (described e.g. in U.S. Pat. Nos. 6,608,055; 7,968,717; and 8,163,913 (Form 11)), crystalline monohydrate (described e.g. in U.S. Pat. Nos. 6,777,423 and 6,908,928) and crystalline solvates (described e.g. in U.S. Pat. No. 7,879,871). The various crystalline forms of tiotropium can be distinguished by a number of different assays, including X-ray Powder Diffraction (XRPD), Differential scanning calorimetry (DSC), crystal structure, and infrared (IR) spectrum analysis. Tiotropium can be synthesized using a variety of methods which are well known in the art (including, e.g. methods described in U.S. Pat. Nos. 6,486,321; 7,491,824; 7,662,963; and 8,344,143).

### SUMMARY

Under certain conditions, a dry powder formulation containing a tiotropium salt and an amino acid (e.g. leucine) result in a decrease of the purity of the tiotropium salt brought about, at least in part, by an increase in tiotropium-related impurities. The impurities are not always present and/or measurable shortly after manufacturing. However, upon storage at room temperature, the impurity levels increase, for example after 3 months, 6 months, 1 year, or 2 years. While removal of amino acid (e.g. leucine) from the formulation might be one way to solve this problem, the amino acid (e.g. leucine) is believed to provide advantages to the respirable dry powders comprising respirable dry particles. These advantages are, for example, improved aerosol performance and powder flowability. A solution is needed that allows for maintaining the amino acid (e.g. leucine) in the formulation with the tiotropium salt without causing a significant growth of impurities of the tiotropium salt and a corresponding decrease in the purity of tiotropium salt during room temperature storage.

To solve the above mentioned problem, acid content was introduced into the dry powder formulation at an effective amount to prevent or delay the formation of the impurities.

A respirable dry powder that contains respirable dry particles that contain a tiotropium salt, one or more amino acids, acid content, sodium chloride, and optionally one or more additional therapeutic agents, where the tiotropium salt is about 0.01% to about 0.5%, the amino acid (e.g., leucine) is about 5% to about 40%, the sodium chloride is about 50% to about 90%, the optional one or more additional therapeutic agents are up to about 30%, and the molar ratio of acid to amino acid is from about 0.002 to about 1, where all percentages are weight percentages on a dry basis and all the components of the respirable dry particles amount to 100%.

A respirable dry powder that contains respirable dry particles that contain a tiotropium salt, one or more amino acids, acid content, sodium chloride, and optionally one or more additional therapeutic agents, where the tiotropium salt is about 0.01% to about 0.5%, the amino acid (e.g., leucine) is about 5% to about 40%, the sodium chloride is about 50% to about 90%, the optional one or more additional therapeutic agents are up to about 30%, and the molar ratio of acid to amino acid is from about 0.002 to about 1, where all percentages are weight percentages on a dry basis and all the components of the respirable dry particles amount to 100%, and where when the respirable dry powder comprising respirable dry particles is sealed in a receptacle and stored for about 12 months at a temperature of about 15° C. to about 30° C., the purity of tiotropium is about 96.0% or greater.

A respirable dry powder that contains respirable dry particles that contain a tiotropium salt, one or more amino acids, acid content, sodium chloride, and optionally one or more additional therapeutic agents, where the tiotropium salt is about 0.01% to about 0.5%, the amino acid (e.g., leucine) is about 5% to about 40%, the sodium chloride is about 50% to about 90%, the optional one or more additional therapeutic agents are up to about 30%, and the molar ratio of acid to amino acid is from about 0.002 to about 1, where all percentages are weight percentages on a dry basis and all the components of the respirable dry particles amount to 100%, and where when the respirable dry powder comprising respirable dry particles is sealed in a receptacle and stored for about 12 months at a temperature of about 15° C. to about 30° C., the amount of tiotropium Impurity B is about 1.0% or less.

A respirable dry powder that contains respirable dry particles that contain a tiotropium salt, one or more amino acids, acid content, sodium chloride, and optionally one or more additional therapeutic agents, where the tiotropium salt is about 0.01% to about 0.5%, the amino acid (e.g., leucine) is about 5% to about 40%, the sodium chloride is about 50% to about 90%, the optional one or more additional therapeutic agents are up to about 30%, and the molar ratio of acid to amino acid is from about 0.002 to about 1, where all percentages are weight percentages on a dry basis and all the components of the respirable dry particles amount to 100%, and where when the respirable dry powder comprising respirable dry particles is sealed in a receptacle and stored for about 12 months at a temperature of about 15° C. to about 30° C., the amount of tiotropium Impurity A is about 1.0% or less.

A respirable dry powder that contains respirable dry particles that contain a tiotropium salt, one or more amino acids, acid content, sodium chloride, and optionally one or more additional therapeutic agents, where the tiotropium salt is about 0.01% to about 0.5%, the amino acid (e.g., leucine) is about 5% to about 40%, the sodium chloride is about 50% to about 90%, the optional one or more additional therapeutic agents are up to about 30%, and the molar ratio of acid to tiotropium is from about 2 to about 1000, where all percentages are weight percentages on a dry basis and all the components of the respirable dry particles amount to 100%.

A respirable dry powder contains respirable dry particles that contain a tiotropium salt, one or more amino acids, acid content, sodium chloride, and optionally one or more additional therapeutic agents, where the tiotropium salt is about 0.01% to about 0.5%, the amino acid (e.g., leucine) is about 5% to about 40%, the sodium chloride is about 50% to about 90%, the optional one or more additional therapeutic agents are up to about 30%, and the molar ratio of acid to tiotropium is from about 2 to about 1000, where all percentages are weight percentages on a dry basis and all the components of the respirable dry particles amount to 100%, and where when the respirable dry powder comprising respirable dry particles is sealed in a receptacle and stored for about 12 months at a temperature of about 15° C. to about 30° C., the purity of tiotropium is about 96.0% or greater.

In one aspect, a dry powder that contains dry particles that contain a tiotropium salt, one or more amino acids, and acid content, where the molar ratio of acid content to amino acid is from about 0.0005 to about 5, or about 0.002 to about 1. In another aspect, a dry powder that contains dry particles that contain a tiotropium salt, one or more amino acids, and acid content, where the molar ratio of acid content to tiotropium is from about 0.5 to about 2000, or about 2 to about 1000. These dry powders may optionally contain a metal cation salt, such as a sodium salt, e.g., sodium chloride. They may also contain one or more additional therapeutic agents. The components in the dry powder may be in any percentage provided that the described molar ratios are maintained. However, the following are examples of weight percentages of the components in the dry powder: the tiotropium salt may be about 0.01% to about 0.5%, the amino acid may be about 5% to about 40%, the optional sodium salt, such as sodium chloride, may be about 50% to about 90%, the optional one or more additional therapeutic agents are up to about 30%, where all percentages are weight percentages on a dry basis and all the components of the dry particles amount to 100%. When the dry powder comprising dry particles is sealed in a receptacle and stored for about 12 months at a temperature of about 15° C. to about 30° C., the stability of the tiotropium may be assessed by any one of the following parameters: the purity of tiotropium is about 96.0% or greater, the amount of tiotropium Impurity B is about 1.0% or less, and/or the amount of tiotropium Impurity A is about 1.0% or less, or any combination.

A method of preparing a stable dry powder tiotropium formulation encompassing spray drying a feedstock to make respirable dry particles, where the feedstock comprises a tiotropium salt, one or more amino acids, acid content, sodium chloride, and optionally one or more additional therapeutic agents, wherein the tiotropium salt is about 0.01% to about 0.5%, the amino acid (e.g., leucine) is about 5% to about 40%, the sodium chloride is about 50% to about 90%, the optional one or more additional therapeutic agents are up to about 30%, and the molar ratio of acid to amino acid is from 0.002 to 1.0, where all percentages are weight percentages on a dry basis and all the components of the respirable dry particles amount to 100%, and sealing a respirable dry powder comprising the respirable dry particles into a receptacle, where when the respirable dry powder comprising respirable dry particles is stored for about 12 months at a temperature of about 15° C. to about 30° C., the purity of tiotropium is about 96.0% or greater.

A method of preparing a stable dry powder tiotropium formulation encompassing spray drying a feedstock to make respirable dry particles, where the feedstock comprises a tiotropium salt, one or more amino acids, acid content, sodium chloride, and optionally one or more additional therapeutic agents, wherein the tiotropium salt is about 0.01% to about 0.5%, the amino acid (e.g., leucine) is about 5% to about 40%, the sodium chloride is about 50% to about 90%, the optional one or more additional therapeutic agents are up to about 30%, and the molar ratio of acid to tiotropium is from 2 to 1000, where all percentages are weight percentages on a dry basis and all the components of the respirable dry particles amount to 100%, and sealing a respirable dry powder comprising the respirable dry particles into a receptacle, where when the respirable dry powder comprising respirable dry particles is stored for about 12 months at a temperature of about 15° C. to about 30° C., the purity of tiotropium is about 96.0% or greater.

For the sake of clarity, the values for the purity of tiotropium, and for the amount of tiotropium Impurity A and tiotropium Impurity B, all refer to values measured at the end of storage, for example, at the end of 12 months.

Some preferred aspects of the respirable dry powder comprising respirable dry particles are as follows. The respirable dry particles comprise an amino acid, a tiotropium salt, acid, and optionally, one or more additional excipients and one or more additional therapeutic agents. The one or more amino acids is preferably leucine, more preferably, L-leucine. The tiotropium salt is preferably selected from the group consisting of tiotropium bromide, tiotropium chloride, and combinations thereof. The acid is preferably selected from the group consisting of hydrochloric acid, hydrobromic acid, nitric acid, sulfuric acid, and combinations thereof; more preferably, hydrochloric acid and/or hydrobromic acid; and most preferably, hydrochloric acid. Alternatively, the acid is selected such that its anion is one that is already present in the formulation and it is a strong acid such that it would be highly dissociated in an aqueous feedstock solution. The one or more optional additional excipients is preferably a salt, more preferably a sodium salt and/or a magnesium salt, more preferably, a sodium salt, and most preferably, sodium chloride. In one aspect, at least one additional excipient is required in the formulation, preferably, sodium chloride. The one or more optional additional therapeutic agents is selected from the group consisting of inhaled corticosteroids (ICS), long-acting beta agonists (LABA), short-acting beta agonists (SABA), anti-inflammatory agents, bifunctional muscarinic antagonist-beta2 agonist (MABA), bronchodilators, or combination thereof. Preferably, the one or more additional therapeutic agent is an ICS, and is preferably, independently selected from the group consisting of fluticasone furoate, mometasone furoate, ciclesonide, and any combination thereof. In one aspect, the optional therapeutic agent is omitted from the formulation.

The one or more amino acids are present in an amount of about 5% to about 40%, about 10% to about 40%, about 12% to about 33%, about 15% to about 25%, or about 19.5% to about 20.5%. The one or more amino acids is preferably leucine, and more preferably L-leucine. The tiotropium salt, preferably tiotropium bromide, tiotropium chloride, or combinations thereof, is present in an amount of about 0.01% to about 0.5%, about 0.02% to about 0.25%, or about 0.05% to about 0.15%. The range of acid content in the dry powders was characterized by the molar ratio of acid content to the amino acid (e.g. leucine) and/or to the tiotropium salt in the dry powder. The molar ratio of acid to leucine in the respirable dry powder was in the range of about 0.0005 to about 5.0, about 0.001 to about 2.0, about 0.002 to about 1, about 0.005 to about 0.5, about 0.01 to about 0.1, or about 0.1 to about 0.5. A preferred ratio is about 0.002 to about 1. The molar ratio of acid to tiotropium in the respirable dry powder was in the range of about 0.5 to about 2000, about 1.0 to about 1000, about 2 to about 1000, about 5 to about 500, about 10 to about 250, about 25 to about 100, or about 100 to about 250. A preferred ratio is about 2 to about 1000. The optional salt, when present, is preferably a sodium salt, and more preferably sodium chloride, and is present in an amount of about 50% to about 90%, about 60% to about 90%, about 67% to about 84%, about 75% to about 82%, about 79.5% to about 80.5%. The additional therapeutic agent, when present, is preferably an ICS. The therapeutic agent is present in an amount up to about 30%, or preferably, about 0.01% to about 15%. Examples of ICSs are fluticasone furoate, mometasone furoate, and ciclesonide. All the percentages are weight percentages on a dry basis and all the components of the respirable dry particles amount to 100%.

The tiotropium purity and impurities can be measured during storage. The respirable dry powder comprising respirable dry particles are packaged and/or stored at a temperature of about 15° C. to about 30° C. They are preferably packaged, e.g., sealed in a receptacle, such that the relative humidity within the receptacle is about 40% or less, about 35% or less, about 30% or less, or about 20% or less; alternatively or in addition, the relative humidity of the environment during sealing the receptacle is about 40% or less, about 35% or less, about 30% or less, or about 20% or less. Alternatively, their relative humidity during packaging is not controlled, but desiccant is included in the packaging to lower the relative humidity during storage. The tiotropium purity and impurity can be measured during storage, e.g., 1 month after packaging, 2 months after packaging, 3 months after packaging, 6 months after packaging, 9 months after packaging, 12 months after packaging, 18 months after packaging, or 24 months after packaging. During storage, the purity of tiotropium is 96.0% or greater, the total amount of Impurities A, B, C, E, F, G and H is 2.0% or less, and/or Impurity A and Impurity B are each 1.0% or less.

In these preferred aspects, the respirable dry powder comprises respirable dry particles that have a volume median geometric diameter (VMGD) of about 10 microns or less, or about 1 microns to about 5 microns; a tap density of greater than 0.4 g/cm³, greater than 0.4 g/cm³ to about 1.2 g/cm³, or about 0.45 g/cm³ to about 1.2 g/cm³; a mass median aerodynamic diameter (MMAD) of between about 1 micron and about 5 microns; a fine particle dose (FPD) less than 5 microns of about 1 microgram to about 5 micrograms, or about 2 micrograms to about 5 micrograms; a FPD less than 4.4 microns of about 1 microgram to about 5 micrograms, or about 2 micrograms to about 5 micrograms; a ratio of the FPD less than 2.0 microns to the FPD less than 5.0 microns of less than 0.25; a ratio of the FPD less than 2.0 microns to the FPD less than 4.4 microns of less than 0.25; a 1/4 bar dispersibility ratio of about 1.5 or less, about 1.4 or less, or about 1.3 or less, as measured by laser diffraction; a 0.5/4 bar dispersibility ratio of about 1.5 or less or about 1.4 or less, as measured by laser diffraction; a fine particle fraction (FPF) of the total dose less than 5.0 of about 35% or more, or preferably, about 50% or more; less than 4.4 microns of about 30% or more, or preferably, about 45% or more; less than 3.0 microns of about 20% or more, or preferably about 30% or more; and/or less than 2.0 microns of 15% or more, or preferably, less than 20% or more; a capsule emitted powder mass (CEPM) of at least 80% when emitted from a passive dry powder inhaler that has a resistance of about 0.036 sqrt(kPa)/liters per minute under the following conditions; an inhalation energy of 2.3 Joules at a flow rate of 30 LPM using a size 3 capsule that contains a total mass of about 10 mg or about 5 mg, said total mass consisting of the respirable dry particles, and wherein the volume median geometric diameter of the respirable dry particles emitted from the inhaler as measured by laser diffraction is about 5 microns or less; or, a CEPM of at least about 80% when emitted from a passive dry powder inhaler that has a resistance of about 0.048 sqrt(kPa)/liters per minute under the following conditions; an inhalation energy of 1.8 Joules at a flow rate of 20 LPM using a size 3 capsule that contains a total mass of about 10 mg or about 5 mg, said total mass consisting of the respirable dry particles, and wherein the volume median geometric diameter of the respirable dry particles emitted from the inhaler as measured by laser diffraction is 5 microns or less.

In these preferred aspects, the respirable dry powder comprising respirable dry particles is used to treat a respiratory disease, or is used to treat or reduce the incidence or severity of an acute exacerbation of a respiratory disease, wherein the respiratory disease is asthma, cystic fibrosis, or non-cystic fibrosis bronchiectasis, or preferably, COPD.

In these preferred aspects, a dry powder inhaler contains the respirable dry powder comprising respirable dry particles, for example, a capsule-based DPI, a blister-based DPI, or a reservoir-based DPI; a receptacle contains the respirable dry powder comprising respirable dry particles, for example, the receptacle is a capsule or a blister; the receptacle contains about 10 mg of the respirable dry powder, or about 5 mg of the respirable dry powder; the receptacle contains a nominal dose of about 6 to about 15 micrograms, about 3 to about 12 micrograms, about 1 to about 6 micrograms, or about 0.5 to about 3 micrograms.

Another aspect of the invention are liquid formulations, which encompass a liquid solution, suspension, emulsion, or slurry containing one or more therapeutic agents such as tiotropium and one or more excipients such as an amino acid, and acid such as a strong acid. A liquid formulation may be a pharmaceutical liquid formulation that is suitable for administration to a patient in need of the therapeutic agent present in the formulation such as tiotropium. A liquid formulation may also be a feedstock liquid formulation that is suitable to be fed into a process that removes the liquid in order to form a dry particles such as respirable dry particles such as by spray drying.

In one aspect, a liquid formulation contains a tiotropium salt, one or more amino acids, and acid content, where the molar ratio of acid content to amino acid is from about 0.0005 to about 5, or about 0.002 to about 1. In another aspect, a liquid formulation contains a tiotropium salt, one or more amino acids, and acid content, where the molar ratio of acid content to tiotropium is from about 0.5 to about 2000, or about 2 to about 1000. The one or more amino acids is preferably leucine and/or glycine. The tiotropium salt is preferably tiotropium bromide, tiotropium chloride, and combinations thereof. The acid is preferably selected from the group consisting of hydrochloric acid, hydrobromic acid, nitric acid, sulfuric acid, and combinations thereof; more preferably, hydrochloric acid and/or hydrobromic acid; and most preferably, hydrochloric acid. Alternatively, the acid is selected such that its anion is one that is already present in the formulation and it is a strong acid such that it would be highly dissociated in an aqueous feedstock solution. The liquid formulation may optionally contain a metal cation salt, such as a sodium salt, e.g., sodium chloride. They may also contain one or more additional therapeutic agents. The components in the liquid formulation may be in any percentage provided that the described molar ratios are maintained. However, the following are examples of weight percentages of the components in the liquid formulation, on a solute or dry basis: the tiotropium salt may be about 0.01% to about 0.5%, the amino acid may be about 5% to about 40%, the optional sodium salt, such as sodium chloride, may be about 50% to about 90%, the optional one or more additional therapeutic agents are up to about 30%, where all percentages are weight percentages on a dry basis and all the components of the dry particles amount to 100%. When the dry powder comprising dry particles is sealed in a receptacle and stored for about 12 months at a temperature of about 15° C. to about 30° C., the stability of the tiotropium may be assessed by any one of the following parameters: the purity of tiotropium is about 96.0% or greater, the amount of tiotropium Impurity B is about 1.0% or less, and/or the amount of tiotropium Impurity A is about 1.0% or less, or any combination.

### DETAILED DESCRIPTION

Some prior formulations of a tiotropium salt and an amino acid (e.g. leucine) that have been spray dried possessed beneficial aerosol properties, low or no amount of impurities of tiotropium, and a high purity of tiotropium salt shortly after manufacturing. However, a problem was discovered by the Inventors that the amount of impurities of the tiotropium salt in those formulations increased over time during storage, making the shelf-life of the product shorter than desired.

Some specific impurities of the tiotropium salt that were monitored were Impurity A (dithienylglycolic acid) and Impurity B (N-demethyl tiotropium). Impurity naming is based on the EUROPEAN PHARMACOPOEIA (Ph. Eur.) Monograph 2420 Tiotropium Bromide Monohydrate, which lists 7 impurities of tiotropium bromide, Impurities A, B, C, E, F, G and H. Impurity B was due to demethylation of the tiotropium salt. Not wishing to be bound by theory, it was speculated based on the examples below that the presence of leucine contributed to the growth of Impurity B in the dry particles during storage. While removal of amino acid (e.g. leucine) from the formulation was one way to solve this problem, the amino acid (e.g. leucine) was believed to provide advantages to the respirable dry powders that comprise respirable dry particles. Therefore another solution was needed for reducing the growth of impurities of the tiotropium salt in the respirable dry powder during storage, including Impurity B.

Factors that have been found by the inventors to affect the growth of impurities of the tiotropium salt during storage were, for example, one of more of the following factors; i) choice of excipients and excipient loading in the dry particles, ii) duration of storage, and iii) environmental conditions such as temperature and relative humidity during bulk powder handling, encapsulation into a dosage form such as a capsule, and/or packaging the dosage form into a storage container such as a blister. Efforts to limit the rise of impurities of the tiotropium salt in the spray dried dry powder formulations proved challenging because a solution that was helpful for reducing one or more of the impurities either did not help reduce the other impurities, caused one or more other impurities to rise, and/or was commercially impractical. For example, reducing the relative humidity during handling, encapsulation and packaging helped to reduce the formation of one impurity but seemed to contribute to the increase of another. Storing the dry powder formulations under refrigeration slowed the growth of all impurities. However, it was neither convenient nor commercially feasible to develop a tiotropium product that requires refrigeration. A product that was stable at room temperature storage, between about 15° C. to 30° C., was desired.

Therefore, the problem of dry powder formulations which contain a tiotropium salt and an amino acid (e.g., leucine) reacting and leading to impurities of the tiotropium salt after being stored as a dry powder for a period of time was solved by introducing acid into a feedstock solution for spray drying in order to provide acid content in the dry powder formulation at an effective amount to prevent or delay the formation of the impurities of tiotropium (e.g., Impurity B), which thereby also contributed to keeping the tiotropium purity high. For the sake of clarity, the feedstock can be a solution, suspension, emulsion or slurry.

Without wishing to be bound by any theory, it is speculated that the protonation of the leucine and/or the tiotropium salt by the acid leads to a reduction in the growth of impurities of tiotropium over time. While any acid known to be safe for delivery to patients as part of a pharmaceutical product is feasible to be used in this invention, a preferred acid is one that highly dissociates in water. A strong acid is most preferred, which is an acid that completely ionizes, i.e., dissociates. Preferred among the strong acids are hydrochloric acid (HCl), hydrobromic acid (HBr), nitric acid, and sulfuric acid. HCl is most preferred because it is known to be safe for delivery to patients as part of a pharmaceutical product, including respiratory products, and the components of the acid are found in the other components of the formulation, so no new components not already in the formulation are added. The range of acid content in the dry powders is characterized by the molar ratio of acid content to the amino acid (e.g. leucine) and/or to the tiotropium salt in the dry powder. The molar ratio of acid to leucine in the respirable dry powder is in the range of about 0.0005 to about 5.0, about 0.001 to about 2.0, about 0.002 to about 1, about 0.005 to about 0.5, about 0.01 to about 0.1, or about 0.1 to about 0.5. A preferred ratio is about 0.002 to about 1. The molar ratio of acid to tiotropium in the respirable dry powder is in the range of about 0.5 to about 2000, about 1.0 to about 1000, about 2 to about 1000, about 5 to about 500, about 10 to about 250, about 25 to about 100, or about 100 to about 250. A preferred ratio is about 2 to about 1000.

### Definitions

The term “acid content” as used herein refers to acid, e.g., hydrochloric acid, present in a dry powder.

The term “dry powder” as used herein refers to a composition that contains finely dispersed respirable dry particles that are capable of being dispersed in an inhalation device and subsequently inhaled by a subject. Such a dry powder may contain up to about 15%, up to about 10%, or up to about 5% water or other solvent, or be substantially free of water or other solvent, or be anhydrous.

The term “dry particles” as used herein refers to respirable particles that may contain up to about 15%, up to about 10%, or up to about 5% water or other solvent, or be substantially free of water or other solvent, or be anhydrous.

The term “respirable” as used herein refers to dry particles or dry powders that are suitable for delivery to the respiratory tract (e.g., pulmonary delivery) in a subject by inhalation. Respirable dry powders or dry particles have a mass median aerodynamic diameter (MMAD) of less than about 10 microns, preferably about 5 microns or less.

The term “liquid formulation” as used herein describes a liquid containing one or more therapeutic agents such as tiotropium, one or more excipients, such as an amino acid, and one or more acids such as hydrochloric acid, as a solution, suspension, emulsion, or slurry. A liquid formulation may be a “pharmaceutical liquid formulation” that is suitable for administration to a patient in need of the therapeutic agent present in the formulation such as tiotropium. A liquid formulation may also be a “feedstock liquid formulation” that is suitable to be fed into a process that removes the liquid in order to form dry particles such as respirable dry particles such as by spray drying.

The term “small” as used herein to describe respirable dry particles refers to particles that have a volume median geometric diameter (VMGD) of about 10 microns or less, preferably about 5 microns or less. VMGD may also be called the volume median diameter (VMD), x50, or Dv50.

As used herein, the terms “administration” or “administering” of respirable dry particles refers to introducing respirable dry particles to the respiratory tract of a subject.

As used herein, the term “respiratory tract” includes the upper respiratory tract (e.g., nasal passages, nasal cavity, throat, and pharynx), respiratory airways (e.g., larynx, trachea, bronchi, and bronchioles) and lungs (e.g., respiratory bronchioles, alveolar ducts, alveolar sacs, and alveoli).

The term “dispersible” is a term of art that describes the characteristic of a dry powder or dry particles to be dispelled into a respirable aerosol. Dispersibility of a dry powder or dry particles is expressed herein as the quotient of the volume median geometric diameter (VMGD) measured at a dispersion (i.e., regulator) pressure of 1 bar divided by the VMGD measured at a dispersion (i.e., regulator) pressure of 4 bar, VMGD at 0.5 bar divided by the VMGD at 4 bar as measured by HELOS/RODOS, VMGD at 0.2 bar divided by the VMGD at 2 bar as measured by HELOS/RODOS, or VMGD at 0.2 bar divided by the VMGD at 4 bar as measured by HELOS/RODOS. These quotients are referred to herein as “1 bar/4 bar,” “0.5 bar/4 bar,” “0.2 bar/2 bar,” and “0.2 bar/4 bar,” respectively, and dispersibility correlates with a low quotient. For example, 1 bar/4 bar refers to the VMGD of respirable dry particles or powders emitted from the orifice of a RODOS dry powder disperser (or equivalent technique) at about 1 bar, as measured by a HELOS or other laser diffraction system, divided by the VMGD of the same respirable dry particles or powders measured at 4 bar by HELOS/RODOS. Thus, a highly dispersible dry powder or dry particles will have a 1 bar/4 bar or 0.5 bar/4 bar ratio that is close to 1.0. Highly dispersible powders have a low tendency to agglomerate, aggregate or clump together and/or, if agglomerated, aggregated or clumped together, are easily dispersed or de-agglomerated as they emit from an inhaler and are breathed in by a subject. Dispersibility can also be assessed by measuring the size emitted from an inhaler as a function of flow rate. VMGD may also be called the volume median diameter (VMD), x50, or Dv50.

The terms “FPF (<X),” “FPF (<X microns),” and “fine particle fraction of less than X microns” as used herein, where X can be, for example, 5.6 microns, 5.0 microns, 4.4 microns, 3.4 microns, 3.0 microns, 2.0 microns, refer to the fraction of a mass of respirable dry particles that have an aerodynamic diameter of less than Y microns, e.g., 2.0 microns, 3.0 microns, 4.4 microns, 5.0 microns. Standard impaction techniques can be used to determine these values, e.g. Andersen Cascade Impactor (ACI), Next Generation Impactor (NGI), etc.

As used herein, the term “emitted dose” or “ED” refers to an indication of the delivery of a drug formulation from a suitable inhaler device after a firing or dispersion event. More specifically, for respirable dry powders comprising respirable dry particles, the ED is a measure of the percentage of powder that is drawn out of a unit dose package and that exits the mouthpiece of an inhaler device. The ED is defined as the ratio of the dose delivered by an inhaler device to the nominal dose (i.e., the mass of powder per unit dose placed into a suitable inhaler device prior to firing). The ED is an experimentally-measured parameter, and can be determined using the method of USP Section 601 Aerosols, Metered-Dose Inhalers and Dry Powder Inhalers, Delivered-Dose Uniformity, Sampling the Delivered Dose from Dry Powder Inhalers, United States Pharmacopeia convention, Rockville, Md., 13^{th} Revision, 222-225, 2007. This method utilizes an in vitro device set up to mimic patient dosing.

The term “capsule emitted powder mass” or “CEPM” as used herein, refers to the amount of dry powder formulation emitted from a capsule or dose unit container during an inhalation maneuver. CEPM is measured gravimetrically, typically by weighing a capsule before and after the inhalation maneuver to determine the mass of powder formulation removed. CEPM can be expressed either as the mass of powder removed, in milligrams, or as a percentage of the initial filled powder mass in the capsule prior to the inhalation maneuver.

The term “effective amount,” as used herein, refers to the amount of active agent needed to achieve the desired therapeutic or prophylactic effect, such as an amount that is sufficient to reduce pathogen (e.g., bacteria, virus) burden, reduce symptoms (e.g., fever, coughing, sneezing, nasal discharge, diarrhea and the like), reduce occurrence of infection, reduce viral replication, or improve or prevent deterioration of respiratory function (e.g., improve forced expiratory volume in 1 second FEV₁ and/or forced expiratory volume in 1 second FEV₁ as a proportion of forced vital capacity FEV₁/FVC, reduce bronchoconstriction), produce an effective serum concentration of a pharmaceutically active agent, increase mucociliary clearance, reduce total inflammatory cell count, or modulate the profile of inflammatory cell counts. The actual effective amount for a particular use can vary according to the particular dry powder or dry particle, the mode of administration, and the age, weight, general health of the subject, and severity of the symptoms or condition being treated. Suitable amounts of dry powders and dry particles to be administered, and dosage schedules for a particular patient can be determined by a clinician of ordinary skill based on these and other considerations.

The term “pharmaceutically acceptable excipient” as used herein means that the excipient can be taken into the lungs with no significant adverse toxicological effects on the lungs. Such excipients are generally regarded as safe (GRAS) by the U.S. Food and Drug Administration.

All references to a therapeutic agent herein includes salt forms, solvates, and stereoisomers.

All references to salts (e.g., sodium containing salts) herein include anhydrous forms and all hydrated forms of the salt.

All weight percentages are given on a dry basis.

### Dry Powders and Dry Particles

Tiotropium Salts

The invention relates to respirable dry powders and respirable dry particles that contain tiotropium as an active ingredient. The chemical structure of tiotropium was first described in U.S. Pat. No. 5,610,163 and RE39,820. Tiotropium salts include salts containing cationic tiotropium with one of the following anions: bromide, fluoride, chloride, iodine, C1-C4-alkylsulphate, sulphate, hydrogen sulphate, phosphate, hydrogen phosphate, di-hydrogen phosphate, nitrate, maleate, acetate, trifluoroacetate, citrate, fumarate, tartrate, oxalate, succinate and benzoate, C1-C4-alkylsulphonate, which may optionally be mono-, di- or tri-substituted by fluorine at the alkyl group, or phenylsulphonate, which may optionally be mono- or poly-substituted by C1-C4-alkyl at the phenyl ring. Tiotropium bromide is an anticholinergic providing therapeutic benefits (e.g., in the treatment of COPD and asthma) and is the active ingredient in SPIRIVA (tiotropium bromide) HANDIHALER (dry powder inhaler) (Boehringer Ingelheim, Germany) Tiotropium bromide is known to crystallize in various forms, such as crystalline anhydrous (described e.g. in U.S. Pat. Nos. 6,608,055; 7,968,717; and 8,163,913 (Form 11)), crystalline monohydrate (described e.g. in U.S. Pat. Nos. 6,777,423 and 6,908,928) and crystalline solvates (described e.g. in U.S. Pat. No. 7,879,871). The various crystalline forms of tiotropium can be distinguished by a number of different assays, including x-ray powder diffraction (XRPD), differential scanning calorimetry (DSC), crystal structure, and infrared (IR) spectrum analysis. Tiotropium can be synthesized using a variety of methods which are well known in the art (including, e.g., methods described in U.S. Pat. Nos. 6,486,321; 7,491,824; 7,662,963; and 8,344,143).

Preferred tiotropium salts include salts containing cationic tiotropium with the following anions: bromide, chloride, and combinations thereof.

Additional Therapeutic Agent

Additional preferred therapeutic combinations with tiotropium include corticosteroids, such as inhaled corticosteroids (ICS), long-acting beta agonists (LABA), short-acting beta agonists (SABA), anti-inflammatory agents, bifunctional muscarinic antagonist-beta2 agonist (MABA), and any combination thereof. In a most preferred embodiment, the tiotropium is combined with one or more ICS. Particularly preferred therapeutic combinations with tiotropium include: a) tiotropium and corticosteroids, such as inhaled corticosteroids (ICS); b) tiotropium and long-acting beta agonists (LABA); c) tiotropium and short-acting beta agonists (SABA); d) tiotropium and anti-inflammatory agents; e) tiotropium and MABA, f) tiotropium and a bronchodilator, or g) combinations thereof, such as tiotropium and ICS and LABA.

Suitable corticosteroids, such as inhaled corticosteroids (ICS), include budesonide, fluticasone, flunisolide, triamcinolone, beclomethasone, mometasone, ciclesonide, dexamethasone, and the like. Tiotropium can be delivered once per day (QD) to patients, so inhaled corticosteroids whose pharmacological data and dosing regimen support administration once per day are preferred. Preferred inhaled corticosteroids are fluticasone, e.g., fluticasone furoate, mometasone, e.g., mometasone furoate, ciclesonide, and the like.

Suitable LABAs include salmeterol, formoterol and isomers (e.g., arformoterol), clenbuterol, tulobuterol, vilanterol (Revolair™), indacaterol, carmoterol, isoproterenol, procaterol, bambuterol, milveterol, olodaterol, and the like.

Suitable SABAs include albuterol, epinephrine, pirbuterol, levalbuterol, metaproteronol, maxair, and the like.

Suitable MABAs include AZD 2115 (AstraZeneca), GSK961081 (GlaxoSmithKline), LAS190792 (Almirall), PF4348235 (Pfizer) and PF3429281 (Pfizer).

Combinations of corticosteroids and LABAs include salmeterol with fluticasone, formoterol with budesonide, formoterol with fluticasone, formoterol with mometasone, indacaterol with mometasone, and the like.

Suitable anti-inflammatory agents include leukotriene inhibitors, phosphodiesterase 4 (PDE4) inhibitors, kinase inhibitors, other anti-inflammatory agents, and the like. Other suitable anti-inflammatory agents can be found in US 2013-0266653, and is hereby incorporated by reference.

Excipients

The respirable dry powders comprising respirable dry particles contain an amino acid. Other acceptable excipients include salts, carbohydrates, sugar alcohols, and the like. Examples of preferred amino acids are non-polar amino acids and polar amino acids, and most preferred non-polar amino acid is leucine. Examples of salts include monovalent or divalent salts such as a sodium salt, a potassium salt, a magnesium salt, a calcium salt, and combinations thereof. Preferred salts are sodium salts and most preferred sodium salt is sodium chloride. Other preferred salts are magnesium salts, calcium salts, or combinations thereof. Examples of carbohydrates are maltodextrin and lactose. An example of a sugar alcohol is mannitol. Other suitable amino acids, carbohydrates, sugar alcohols, and monovalent salts can be found in US 2013-0266653, and other suitable monovalent salts can be found in US 2013-0266653, and both are hereby incorporated by reference.

Other suitable salts include divalent salts and can be found in US 2012-0064126 and US 2013-0213398, and both are hereby incorporated by reference.

Acid Content

The term “acid content” refers to acid present in a dry powder. Acids that are suitable for use in this invention are pharmaceutically acceptable acids. Preferred acids are strong acids such that they would be highly disassociated in an aqueous feedstock solution. Some examples of such acids are hydrochloric acid, hydrobromic acid, nitric acid, and sulfuric acid. Also preferred are those which the anion is one that is already present in the formulation, e.g., hydrochloric acid when sodium chloride and/or tiotropium chloride are used, or hydrobromic acid when tiotropium bromide is used in the formulation. For example, when the dry powder comprises sodium chloride and/or tiotropium chloride, the preferred acid is hydrochloric acid because it is a strong acid and the chloride ion is present in the dry powder.

Impurities

Impurity is defined herein according to ICH HARMONISED TRIPARTITE GUIDELINE IMPURITIES IN NEW DRUG PRODUCTS Q3B(R2) as any component of a drug product that is not the drug substance or an excipient in the drug product. Specified impurities of tiotropium bromide are A, B, C, E, F, G and H, as outlined in Ph. Eur. Monograph 2420 Tiotropium Bromide Monohydrate, and listed in Table 1. Non-specified impurities are referred to as unknown impurities.

TABLE 1
Identity of Specified Tiotropium Bromide Impurities

| Specified Impurity | Impurity Name |
|---|---|
| A | 2-hydroxy-2,2-dithiophen-2-ylacetic acid |
| B | (1R,2R,4S,5S,7s)-9-methyl-3-oxa-9- |
| | azatricyclo[3.3.1.0^{2,4}]nonan-7-yl 2-hydroxy- |
| | 2,2-dithiophen-2-ylacetate |
| C | (1R,3s,5S)-3-[(2-hydroxy-2,2-dithiophen-2- |
| | ylacetyl)oxy]8-,8-dimethyl-8- |
| | azoniabicyclo[3.2.1]oct-6-ene bromide |
| E | Methyl 2-hydroxy-2,2-dithiophen-2-ylacetate |
| F | Dithiophen-2-ylmethanone |
| G | (1R,2R,4S,5S,7s)-7-hydroxy-9,9-dimethyl-3- |
| | oxa-9-azoniatricyclo[3.3.1.0^{2,4]}nonane bromide |
| H | (1s,3RS,4RS,5RS,7SR)-4-hydroxy-6,6- |
| | dimethyl-2-oxa-6- |
| | azoniatricyclo[3.3.1.0^{3,7}]nonane bromide |

Two of the impurities found to form during the storage of the respirable dry powders comprising respirable dry particles containing the tiotropium salt and the amino acid are Impurity A (dithienylglycolic acid) and Impurity B (N-demethyl tiotropium). Impurity naming is based on the EUROPEAN PHARMACOPOEIA (Ph. Eur.) Monograph 2420 Tiotropium Bromide Monohydrate, which lists seven impurities of tiotropium bromide, Impurities A, B, C, E, F, G and H. Impurities A and G are believed to be the product of a hydrolysis reaction of the tiotropium salt, and Impurity B is believed to be due to the demethylation of the tiotropium salt. Many of these impurities, e.g., Impurity A, Impurity B, can also be formed by degradation of other tiotropium salts, e.g., tiotropium chloride.

Ranges

The respirable dry powders comprise respirable dry particles which comprise at least a tiotropium salt, an amino acid, and acid content. The preferred tiotropium salt is selected from the group consisting of tiotropium bromide, tiotropium chloride, and combinations thereof. The amino acid is preferably leucine. The acid content is preferably a strong acid, such as hydrochloric acid, hydrobromic acid, nitric acid or sulfuric acid, and most preferably hydrochloric acid. The respirable dry powders that comprise respirable dry particles can also comprise other components as well. For example, respirable dry powders that comprise respirable dry particles contain a salt as an excipient. Preferred salts are selected from the group consisting of sodium salts, magnesium salts, calcium salts, potassium salts, and combinations thereof. More preferred salts are sodium salts. The most preferred salt is sodium chloride. The formulation may also contain one or more additional therapeutic agents.

The components of the respirable dry powder formulation preferably have the following amounts. The tiotropium salt is about 0.01% to about 0.5%, about 0.02% to about 0.25%, or about 0.05% to about 0.15%. The amino acid is about 5% to about 40%, about 10% to about 40%, about 12% to about 33%, about 15% to about 25%, or about 19.5% to about 20.5%. The range of acid content in the dry powders was characterized by the molar ratio of acid content to the amino acid (e.g. leucine) and/or to the tiotropium salt in the dry powder. The molar ratio of acid to leucine in the respirable dry powder was in the range of about 0.0005 to about 5.0, about 0.001 to about 2.0, about 0.002 to about 1, about 0.005 to about 0.5, about 0.01 to about 0.1, or about 0.1 to about 0.5. A preferred ratio is about 0.002 to about 1. The molar ratio of acid to tiotropium in the respirable dry powder was in the range of about 0.5 to about 2000, about 1.0 to about 1000, about 2 to about 1000, about 5 to about 500, about 10 to about 250, about 25 to about 100, or about 100 to about 250. A preferred ratio is about 2 to about 1000. The salt is preferably sodium chloride and is about 50% to about 90%, about 60% to about 90%, about 67% to about 84%, about 75% to about 82%, or about 79.5% to about 80.5%. The one or more optional additional therapeutic agents, when present, is present up to about 30%, about 0.001% to about 20%, or about 0.01% to about 10%.

The respirable dry powder comprising respirable dry particles are packaged and stored at a temperature of about 15° C. to about 30° C. They are preferably packaged, e.g., sealed in a receptacle, such that the relative humidity within the receptacle is about 40% or less, about 35% or less, about 30% or less, or about 20% or less. Alternatively or in addition, the relative humidity of the environment during sealing the receptacle is about 40% or less, about 35% or less, about 30% or less, or about 20% or less. Alternatively, the relative humidity during packaging is not controlled, but desiccant is included in the packaging to lower the relative humidity during storage. The tiotropium purity and/or impurities can be measured during storage, e.g., 1 month after packaging, 2 months after packaging, 3 months after packaging, 6 months after packaging, 9 months after packaging, 12 months after packaging, 18 months after packaging, or 24 months after packaging. During storage, the purity of each therapeutic agent is 96.0% or greater, the total amount of Impurities A, B, C, E, F, G and H is 2.0% or less, and/or Impurity A and Impurity B are each 1.0% or less.

Additional ranges during storage for the purity of tiotropium are 97.0% or greater, 98.0% or greater, or 99.0% or greater. Additional ranges for the total amount of Impurities A, B, C, E, F, G and H are 1.5% or less, 1.0% or less, or 0.5% or less, and/or Impurity A and Impurity B are each 0.75% or less, each 0.5% or less, or each 0.25% or less.

All the percentages are weight percentages on a dry basis and all the components of the respirable dry particles amount to 100%.

Aerosol Properties

The respirable dry powders and/or respirable dry particles are preferably small, dense in mass, and dispersible. To measure volumetric median geometric diameter (VMGD), a laser diffraction system may be used, e.g., a Spraytec system (particle size analysis instrument, Malvern Instruments) or a HELOS/RODOS system (laser diffraction sensor with dry dispensing unit, Sympatec GmbH). The respirable dry particles have a VMGD as measured by laser diffraction at the dispersion pressure setting of 1.0 bar using a HELOS/RODOS system of about 10 microns or less (e.g., about 0.5 μm to about 10 μm), about 5 microns or less (e.g., about 0.5 μm to about 5 μm), about 4 μm or less (e.g., about 0.5 μm to about 4 μm), about 3 μm or less (e.g., about 0.5 μm to about 3 μm), about 1 μm to about 5 μm, about 1 μm to about 4 μm. Preferably the VMGD is about 5 microns or less (e.g., about 1 μm to about 5 μm), or about 4 μm or less (e.g., about 1 μm to about 4 μm).

The respirable dry powders and/or respirable dry particles have 1 bar/4 bar and/or 0.5 bar/4 bar ratio of less than about 2.0 (e.g., about 0.9 to less than about 2), about 1.7 or less (e.g., about 0.9 to about 1.7) about 1.5 or less (e.g., about 0.9 to about 1.5), about 1.4 or less (e.g., about 0.9 to about 1.4), or about 1.3 or less (e.g., about 0.9 to about 1.3), and preferably have a 1 bar/4 bar and/or a 0.5 bar/4 bar of about 1.5 or less (e.g., about 1.0 to about 1.5), and/or about 1.4 or less (e.g., about 1.0 to about 1.4).

The respirable dry powders and/or respirable dry particles have a tap density of greater than 0.4 g/cm³ (e.g., greater than 0.4 g/cm³ to about 1.2 g/cm³), at least about 0.45 g/cm³ (e.g., about 0.45 g/cm³ to about 1.2 g/cm³), at least about 0.5 g/cm³ (e.g., about 0.5 g/cm³ to about 1.2 g/cm³), at least about 0.55 g/cm³ (e.g., about 0.55 g/cm³ to about 1.2 g/cm³), at least about 0.6 g/cm³ (e.g., about 0.6 g/cm³ to about 1.2 g/cm³), or at least about 0.6 g/cm³ to about 1.0 g/cm³.

The respirable dry powders and/or respirable dry particles have an MMAD of less than 10 microns (e.g., about 0.5 microns to less than 10 microns), preferably an MMAD of about 5 microns or less (e.g., about 1 micron to about 5 microns), about 2 microns to about 5 microns, or about 2.5 microns to about 4.5 microns. In a preferred embodiment, the MMAD is measured using a capsule based passive dry powder inhaler such as the RS01 UHR2 (RS01 Model 7, Ultrahigh resistance 2 (UHR2) Plastiape S.p.A.), which had specific resistance of 0.048 sqrt(kPa)/liters per minute, and as measured at 39 LPM, the MMAD range is about 1.0 micron to about 5.0 microns, or a preferred MMAD range is about 3.0 microns to about 5.0 microns, or about 3.8 microns to about 4.3 microns. In another preferred embodiment, the MMAD is measured using a capsule based passive dry powder inhaler such as the RS01 Model 7, High resistance (HR), Plastiape S.p.A., which had specific resistance of 0.036 sqrt(kPa)/liters per minute, and as measured at 60 LPM the MMAD range is about 1.0 micron to about 5.0 microns, or a preferred MMAD range is about 2.9 microns to about 4.0 microns, or about 2.9 microns to about 3.5 microns.

The respirable dry powders and/or respirable dry particles have an FPF of less than about 5.6 microns (FPF<5.6 μm) of the total dose of at least about 35%, preferably at least about 45%, at least about 60%, between about 45% to about 80%, or between about 60% to about 80%. In addition, the respirable dry powders and/or respirable dry particles have a FPF of less than about 3.4 microns (FPF<3.4 μm) of the total dose of at least about 20%, preferably at least about 25%, at least about 30%, at least about 40%, between about 25% to about 60%, or between about 40% to about 60%.

The respirable dry powders and/or respirable dry particles have a FPD of less than about 5.0 microns (FPD<5.0 μm) and/or less than about 4.4 microns (FPD<5.0 μm) as a percentage of the total dose of at least 30%, at least 35%, at least 40%, at least 45%, at least 50%, at least 55%, or at least 60%. Alternatively, the FPD<5.0 μm or FPD<4.40 μm for tiotropium is about 1 microgram to about 5 micrograms, or about 2 micrograms to about 5 micrograms. The ratio of the FPD less than 2.0 microns to the FPD less than 5.0 microns or the FPD less than 2.0 microns to the FPD less than 4.4 microns is less than 0.25.

In some aspects, the invention provides a method of efficiently delivering a dose of tiotropium as a dry powder. The efficiency of delivering a dose of tiotropium can be characterized based on delivering an effective amount of tiotropium to the lungs with a lower nominal dose filled into the capsule than from a standard dry powder formulation such as SPIRIVA (tiotropium bromide) HANDIHALER (dry powder inhaler) which has a nominal dose of 18 micrograms of tiotropium. The efficiency of delivering a dose of tiotropium can further be characterized by delivering a fine particle dose similar to that of a capsule of SPIRIVA (tiotropium bromide) HANDIHALER (dry powder inhaler) with a lower nominal dose filled into the capsule. The efficiency of delivering a dose of tiotropium can further be characterized by delivering a fine particle dose less than about 4.4 microns (FPD<4.4 μm) similar to that of a capsule of SPIRIVA (tiotropium bromide) HANDIHALER (dry powder inhaler) with a lower nominal dose filled into the capsule.

The efficiency of delivering a dose of tiotropium can be further characterized in an aspect of the current invention based on delivering an effective amount of tiotropium to the lungs to achieve a similar improvement in lung function, preferably, a similar change in forced expiratory volume in one second (FEV₁), or, more preferably, a similar change in trough FEV₁ response at steady state as SPIRIVA (tiotropium bromide) HANDIHALER (dry powder inhaler), but with a lower nominal dose than SPIRIVA (tiotropium bromide) HANDIHALER (dry powder inhaler). In one aspect, when the nominal dose of tiotropium in the respirable dry powders and/or respirable dry particles is 70% or less, 50% or less, or preferably 35% or less, 25% or less, or 20% or less, 15% or less, 10% or less, or 5% or less of the nominal dose of SPIRIVA (tiotropium bromide) HANDIHALER (dry powder inhaler), which is 18 micrograms of tiotropium; the change in FEV₁ is about 80% or greater of the change in FEV₁ observed in patients taking SPIRIVA (tiotropium bromide) HANDIHALER (dry powder inhaler), preferably, about 85% or greater of the change in FEV₁ observed in patients taking SPIRIVA (tiotropium bromide) HANDIHALER (dry powder inhaler), more preferably, 90% or greater of the change in FEV₁ observed for patients taking SPIRIVA (tiotropium bromide) HANDIHALER (dry powder inhaler), or most preferably, about 95% or greater of the change in FEV₁ observed in patients taking SPIRIVA (tiotropium bromide) HANDIHALER (dry powder inhaler).

In another aspect, when the nominal dose of tiotropium in the respirable dry powders and/or respirable dry particles is 70% or less, 50% or less, or preferably 35% or less, 25% or less; or, 20% or less, 15% or less, 10% or less, or 5% or less of the nominal dose of SPIRIVA (tiotropium bromide) HANDIHALER (dry powder inhaler), which is 18 micrograms of tiotropium; the change in trough FEV₁ response at steady state is about 80 mL or greater, about 90 mL or greater, preferably about 100 mL or greater, about 110 mL or greater, about 120 mL or greater.

The respirable dry powders and/or respirable dry particles can be contained in a receptacle that may contain about 15 mg, 10 mg, 7.5 mg, 5 mg, 2.5 mg, or 1 mg of mass of the respirable dry powder and/or respirable dry particles. Such receptacles may contain a nominal dose of tiotropium that ranges between about 3 to about 12 micrograms, between about 3 to about 9 micrograms, between about 3 to about 6 micrograms, between about 1.5 to about 12 micrograms, between about 0.5 to about 6 micrograms, between about 0.5 to about 3 micrograms and between about 1 to about 3 micrograms. In certain embodiments, the receptacle may contain a nominal dose of tiotropium of about 0.5 micrograms, about 1 microgram, about 1.5 micrograms, about 2 micrograms, about 2.5 micrograms, 3 micrograms, about 6 micrograms, about 9 micrograms, or about 12 micrograms. The receptacle can be contained in a dry powder inhaler or can be packaged and/or sold separately.

The respirable dry powders and/or respirable dry particles can have a water or solvent content of up to about 15% by weight of the respirable dry powder or particle. For example, the water or solvent content is up to about 10%, up to about 5%, or preferably between about 0.1% and about 3%, between about 0.01% and 1%, or be substantially free of water or other solvent, or be anhydrous.

The respirable dry powders and/or respirable dry particles can be administered with low inhalation energy. In order to relate the dispersion of powder at different inhalation flow rates, volumes, and from inhalers of different resistances, the energy required to perform the inhalation maneuver can be calculated. Inhalation energy can be calculated from the equation E=R²Q²V where E is the inhalation energy in Joules, R is the inhaler resistance in kPa^{1/2}/LPM, Q is the steady flow rate in L/min and V is the inhaled air volume in L.

The respirable dry powders and/or respirable dry particles are characterized by a high emitted dose (e.g., CEPM of at least about 75%, at least about 80%, at least about 85%, at least about 90%, at least about 95%) from a dry powder inhaler when a total inhalation energy of about 5 Joules, about 3.5 Joules, about 2.3 Joules, about 1.8 Joules, about 1 Joule, about 0.8 Joule, about 0.5 Joule, or about 0.3 Joule is applied to the dry powder inhaler.

In one aspect, the respirable dry powders and/or respirable dry particles are characterized by a capsule emitted powder mass of at least about 80% when emitted from a passive dry powder inhaler that has a resistance of about 0.036 sqrt(kPa)/liters per minute under the following conditions: an inhalation energy of about 2.3 Joules at a flow rate of 30 LPM using a size 3 capsule that contains a total mass of about 10 mg, or about 5 mg, the total mass consisting of the respirable dry powders and/or respirable dry particle, and wherein the volume median geometric diameter of the respirable dry particles emitted from the inhaler is 5 microns or less.

In one aspect, the respirable dry powders and/or respirable dry particles are characterized by a capsule emitted powder mass of at least about 80% when emitted from a passive dry powder inhaler that has a resistance of about 0.048 sqrt(kPa)/liters per minute under the following conditions: an inhalation energy of about 1.8 Joules at a flow rate of 20 LPM using a size 3 capsule that contains a total mass of about 10 mg, or about 5 mg, the total mass consisting of the respirable dry powders and/or respirable dry particle, and wherein the volume median geometric diameter of the respirable dry particles emitted from the inhaler is 5 microns or less.

Healthy adult populations are predicted to be able to achieve inhalation energies ranging from 2.9 Joules for comfortable inhalations to 22 Joules for maximum inhalations by using values of peak inspiratory flow rate (PIFR) measured by Clarke et al. (Journal of Aerosol Med, 6(2), p.99-110, 1993) for the flow rate Q from two inhaler resistances of 0.02 and 0.055 kPa^{1/2}/LPM, with an inhalation volume of 2 L based on both FDA guidance documents for dry powder inhalers and on the work of Tiddens et al. (Journal of Aerosol Med, 19(4), p.456-465, 2006) who found adults averaging 2.2 L inhaled volume through a variety of DPIs.

Mild, moderate and severe adult COPD patients are predicted to be able to achieve maximum inhalation energies of 5.1 to 21 Joules, 5.2 to 19 Joules, and 2.3 to 18 Joules respectively. This is again based on using measured PIFR values for the flow rate Q in the equation for inhalation energy. The PIFR achievable for each group is a function of the inhaler resistance that is being inhaled through. The work of Broeders et al. (Eur Respir J, 18, p.780-783, 2001) was used to predict maximum and minimum achievable PIFR through two dry powder inhalers of resistances 0.021 and 0.032 kPa^{1/2}/LPM for each.

Similarly, adult asthmatic patients are predicted to be able to achieve maximum inhalation energies of 7.4 to 21 Joules based on the same assumptions as the COPD population and PIFR data from Broeders et al.

Healthy adults and children, COPD patients, asthmatic patients ages 5 and above, and CF patients, for example, are capable of providing sufficient inhalation energy to empty and disperse the Respirable dry powders comprising respirable dry particles of the invention.

An advantage of the invention is the production of powders that disperse well across a wide range of flow rates and are relatively flowrate independent. The respirable dry powder and/or respirable dry particles of the invention enable the use of a simple, passive DPI for a wide patient population.

Methods for Preparing Dry Powders and Dry Particles

The respirable dry particles and dry powders can be prepared using any suitable method. Many suitable methods for preparing respirable dry powders and/or respirable dry particles are conventional in the art, and include single and double emulsion solvent evaporation, spray drying, spray-freeze drying, milling (e.g., jet milling), blending, solvent extraction, solvent evaporation, phase separation, simple and complex coacervation, interfacial polymerization, suitable methods that involve the use of supercritical carbon dioxide (CO₂), sonocrystalliztion, nanoparticle aggregate formation and other suitable methods, including combinations thereof. Respirable dry particles can be made using methods for making microspheres or microcapsules known in the art. These methods can be employed under conditions that result in the formation of respirable dry particles with desired aerodynamic properties (e.g., aerodynamic diameter and geometric diameter). If desired, respirable dry particles with desired properties, such as size and density, can be selected using suitable methods, such as sieving.

Suitable methods for selecting respirable dry particles with desired properties, such as size and density, include wet or dry sieving, dry sieving, and aerodynamic classifiers (such as cyclones).

The respirable dry particles are preferably spray dried. Suitable spray-drying techniques are described, for example, by K. Masters in “Spray Drying Handbook”, John Wiley & Sons, New York (1984). Generally, during spray-drying, heat from a hot gas such as heated air or nitrogen is used to evaporate a solvent from droplets formed by atomizing a continuous liquid feed. When hot air is used, the moisture in the air is at least partially removed before its use. When nitrogen is used, the nitrogen gas can be run “dry”, meaning that no additional water vapor is combined with the gas. If desired the moisture level of the nitrogen or air can be set before the beginning of spray dry run at a fixed value above “dry” nitrogen. If desired, the spray drying or other instruments, e.g., jet milling instrument, used to prepare the dry particles can include an inline geometric particle sizer that determines a geometric diameter of the respirable dry particles as they are being produced, and/or an inline aerodynamic particle sizer that determines the aerodynamic diameter of the respirable dry particles as they are being produced.

For spray drying, solutions, emulsions or suspensions that contain the components of the dry particles to be produced in a suitable solvent (e.g., aqueous solvent, organic solvent, aqueous-organic mixture or emulsion) are distributed to a drying vessel via an atomization device. For example, a nozzle or a rotary atomizer may be used to distribute the solution or suspension to the drying vessel. The nozzle can be a two-fluid nozzle, which is in an internal mixing setup or an external mixing setup. Alternatively, a rotary atomizer having a 4- or 24-vaned wheel may be used. Examples of suitable spray dryers that can be outfitted with either a rotary atomizer or a nozzle, include, a Mobile Minor Spray Dryer or the Model PSD-1, both manufactured by GEA Niro, Inc. (Denmark). Actual spray drying conditions will vary depending, in part, on the composition of the spray drying solution or suspension and material flow rates. The person of ordinary skill will be able to determine appropriate conditions based on the compositions of the solution, emulsion or suspension to be spray dried, the desired particle properties and other factors. In general, the inlet temperature to the spray dryer is about 90° C. to about 300° C., and preferably is about 180° C. to about 285° C. Another preferable range is between 130° C. to about 200° C. The spray dryer outlet temperature will vary depending upon such factors as the feed temperature and the properties of the materials being dried. Generally, the outlet temperature is about 50° C. to about 150° C., preferably about 90° C. to about 120° C., or about 98° C. to about 108° C. Another preferable range is between 40° C. to about 110° C., preferably about 50° C. to about 90° C. If desired, the respirable dry particles that are produced can be fractionated by volumetric size, for example, using a sieve, or fractioned by aerodynamic size, for example, using a cyclone, and/or further separated according to density using techniques known to those of skill in the art.

Additional examples of spray dryers include the ProCepT Formatrix R&D spray dryer (ProCepT nv, Zelzate, Belgium). BUCHI B-290 MINI SPRAY DRYER (BUCHI Labortechnik AG, Flawil, Switzerland). An additional preferred range for the inlet temperature to the spray dryer is about 180° C. to about 285° C. An additional preferred range for the outlet temperature from the spray dryer is about 40° C. to about 110° C., more preferably about 50° C. to about 90° C.

To prepare the respirable dry particles of the invention, generally, a solution, emulsion or suspension that contains the desired components of the dry powder (i.e., a feed stock) is prepared and spray dried under suitable conditions. Preferably, the dissolved or suspended solids concentration in the feed stock is at least about 1 g/L, at least about 2 g/L, at least about 5 g/L, at least about 10 g/L, at least about 15 g/L, at least about 20 g/L, at least about 30 g/L, at least about 40 g/L, at least about 50 g/L, at least about 60 g/L, at least about 70 g/L, at least about 80 g/L, at least about 90 g/L, or at least about 100 g/L. The feed stock can be provided by preparing a single solution or suspension by dissolving or suspending suitable components (e.g., salts, excipients, other active ingredients) in a suitable solvent. The solvent, emulsion or suspension can be prepared using any suitable methods, such as bulk mixing of dry and/or liquid components or static mixing of liquid components to form a combination. For example, a hydrophilic component (e.g., an aqueous solution) and a hydrophobic component (e.g., an organic solution) can be combined using a static mixer to form a combination. The combination can then be atomized to produce droplets, which are dried to form respirable dry particles. Preferably, the atomizing step is performed immediately after the components are combined in the static mixer. Alternatively, the atomizing step is performed on a bulk mixed solution.

The feed stock, or components of the feed stock, can be prepared using any suitable solvent, such as an organic solvent, an aqueous solvent or mixtures thereof. Suitable organic solvents that can be employed include but are not limited to alcohols such as, for example, ethanol, methanol, propanol, isopropanol, butanols, and others. Other organic solvents include but are not limited to perfluorocarbons, dichloromethane, chloroform, ether, ethyl acetate, methyl tert-butyl ether and others. Co-solvents that can be employed include an aqueous solvent and an organic solvent, such as, but not limited to, the organic solvents as described above. Aqueous solvents include water and buffered solutions.

Respirable dry particles and dry powders can be fabricated and then separated, for example, by filtration or centrifugation by means of a cyclone, to provide a particle sample with a preselected size distribution. For example, greater than about 30%, greater than about 40%, greater than about 50%, greater than about 60%, greater than about 70%, greater than about 80%, or greater than about 90% of the respirable dry particles in a sample can have a diameter within a selected range. The selected range within which a certain percentage of the respirable dry particles fall can be, for example, any of the size ranges described herein, such as between about 0.1 to about 3 microns VMGD.

The feed stock or components of the feed stock can have any desired pH, viscosity or other properties. If desired, a pH buffer can be added to the solvent or co-solvent or to the formed mixture. Generally, the pH of the mixture ranges from about 2 to about 5.

The diameter of the respirable dry particles, for example, their VMGD, can be measured using an electrical zone sensing instrument such as a Multisizer IIe (Coulter Electronic, Luton, Beds, England), or a laser diffraction instrument such as a HELOS system (Sympatec, Princeton, N.J.) or a Mastersizer system (Malvern, Worcestershire, UK). Other instruments for measuring particle geometric diameter are well known in the art. The diameter of respirable dry particles in a sample will range depending upon factors such as particle composition and methods of synthesis. The distribution of size of respirable dry particles in a sample can be selected to permit optimal deposition within targeted sites within the respiratory system.

Experimentally, aerodynamic diameter can be determined using time of flight (TOF) measurements. For example, an instrument such as the Aerosol Particle Sizer (APS) Spectrometer (TSI Inc., Shoreview, Minn.) can be used to measure aerodynamic diameter. The APS measures the time taken for individual respirable dry particles to pass between two fixed laser beams.

Aerodynamic diameter also can be experimentally determined directly using conventional gravitational settling methods, in which the time required for a sample of respirable dry particles to settle a certain distance is measured. Indirect methods for measuring the mass median aerodynamic diameter include the Andersen Cascade Impactor (ACI), next generation impactor (NGI), and the multi-stage liquid impinger (MSLI) methods. The methods and instruments for measuring particle aerodynamic diameter are well known in the art.

Tap density is a measure of the envelope mass density characterizing a particle. Tap density is accepted in the field as an approximation of the envelope mass density of a particle. The envelope mass density of a particle of a statistically isotropic shape is defined as the mass of the particle divided by the minimum sphere envelope volume within which it can be enclosed. Features which can contribute to low tap density include irregular surface texture, high particle cohesiveness and porous structure. Tap density can be measured by using instruments known to those skilled in the art such as the Dual Platform Microprocessor Controlled Tap Density Tester (Vankel, N.C.), a GeoPyc™ instrument (Micrometrics Instrument Corp., Norcross, Ga.), or SOTAX Tap Density Tester model TD2 (SOTAX Corp., Horsham, Pa.). Tap density can be determined using the method of USP Bulk Density and Tapped Density, United States Pharmacopeia convention, Rockville, Md., 10^{th} Supplement, 4950-4951, 1999.

Fine particle fraction can be used as one way to characterize the aerosol performance of a dispersed powder. Fine particle fraction describes the size distribution of airborne respirable dry particles. Gravimetric analysis, using a Cascade impactor, is one method of measuring the size distribution, or fine particle fraction, of airborne respirable dry particles. The Andersen Cascade Impactor (ACI) is an eight-stage impactor that can separate aerosols into nine distinct fractions based on aerodynamic size. The size cutoffs of each stage are dependent upon the flow rate at which the ACI is operated. The ACI is made up of multiple stages consisting of a series of nozzles (i.e., a jet plate) and an impaction surface (i.e., an impaction disc). At each stage an aerosol stream passes through the nozzles and impinges upon the surface. Respirable dry particles in the aerosol stream with a large enough inertia will impact upon the plate. Smaller respirable dry particles that do not have enough inertia to impact on the plate will remain in the aerosol stream and be carried to the next stage. Each successive stage of the ACI has a higher aerosol velocity in the nozzles so that smaller respirable dry particles can be collected at each successive stage. Specifically, an eight-stage ACI is calibrated so that the fraction of powder that is collected on stage 2 and all lower stages including the final collection filter is composed of respirable dry particles that have an aerodynamic diameter of less than 4.4 microns. The airflow at such a calibration is approximately 60 L/min.

If desired, a two-stage collapsed ACI can also be used to measure fine particle fraction. The two-stage collapsed ACI consists of only stages 0 and 2 of the eight-stage ACI, as well as the final collection filter, and allows for the collection of two separate powder fractions. Specifically, a two-stage collapsed ACI is calibrated so that the fraction of powder that is collected on stage two is composed of respirable dry particles that have an aerodynamic diameter of less than 5.6 microns and greater than 3.4 microns. The fraction of powder passing stage two and depositing on the final collection filter is thus composed of respirable dry particles having an aerodynamic diameter of less than 3.4 microns. The airflow at such a calibration is approximately 60 L/min.

The FPF (<5.6) has been demonstrated to correlate to the fraction of the powder that is able to make it into the lungs of the patient, while the FPF (<3.4) has been demonstrated to correlate to the fraction of the powder that reaches the deep lung of a patient. These correlations provide a quantitative indicator that can be used for particle optimization.

Emitted dose can be determined using the method of USP Section 601 Aerosols, Metered-Dose Inhalers and Dry Powder Inhalers, Delivered-Dose Uniformity, Sampling the Delivered Dose from Dry Powder Inhalers, United States Pharmacopeia convention, Rockville, Md., 13^{th} Revision, 222-225, 2007. This method utilizes an in vitro device set up to mimic patient dosing.

An ACI can be used to approximate the emitted dose, which herein is called gravimetric recovered dose and analytical recovered dose. “Gravimetric recovered dose” is defined as the ratio of the powder weighed on all stage filters of the ACI to the nominal dose. “Analytical recovered dose” is defined as the ratio of the powder recovered from rinsing all stages, all stage filters, and the induction port of the ACI to the nominal dose.

Another way to approximate emitted dose is to determine how much powder leaves its container, e.g. capsule or blister, upon actuation of a dry powder inhaler (DPI). This takes into account the percentage leaving the capsule, but does not take into account any powder depositing on the DPI. The emitted powder mass is the difference in the weight of the capsule with the dose before inhaler actuation and the weight of the capsule after inhaler actuation. This measurement can be called the capsule emitted powder mass (CEPM) or sometimes termed “shot-weight”.

A Multi-Stage Liquid Impinger (MSLI) is another device that can be used to measure fine particle fraction. The Multi-Stage Liquid Impinger operates on the same principles as the ACI, although instead of eight stages, MSLI has five. Additionally, each MSLI stage consists of an ethanol-wetted glass frit instead of a solid plate. The wetted stage is used to prevent particle bounce and re-entrainment, which can occur when using the ACI.

The Next Generation Pharmaceutical Impactor (NGI) is another device that can be used to measure fine particle fraction. The NGI is an eight-stage impactor that can separate aerosols into nine distinct fractions based on aerodynamic size. The size cutoffs of each stage are dependent upon the flow rate at which the NGI is operated. The NGI is made up of multiple stages consisting of a series of nozzles (i.e., a jet plate) and an impaction surface (i.e., an impaction disc). At each stage an aerosol stream passes through the nozzles and impinges upon the surface. Respirable dry particles in the aerosol stream with a large enough inertia will impact upon the plate. Smaller respirable dry particles that do not have enough inertia to impact on the plate will remain in the aerosol stream and be carried to the next stage. Each successive stage of the NGI has a higher aerosol velocity in the nozzles so that smaller respirable dry particles can be collected at each successive stage. Specifically, an eight-stage NGI is calibrated so that the fraction of powder that is collected on stage 2 and all lower stages including the final collection filter is composed of respirable dry particles that have an aerodynamic diameter of less than 4.4 microns. The airflow at such a calibration is approximately 60 L/min.

The geometric particle size distribution can be measured for the respirable dry powder after being emitted from a dry powder inhaler (DPI) by use of a laser diffraction instrument such as the Malvern Spraytec. With the inhaler mounted in the open-bench configuration, an airtight seal is made to the air inlet side of the DPI, causing the outlet aerosol to pass perpendicularly through the laser beam as an external flow. In this way, known flow rates can be blown through the DPI by positive pressure to empty the DPI. The resulting geometric particle size distribution of the aerosol is measured by the photodetectors with samples typically taken at 1000 Hz for the duration of the inhalation and the Dv50, GSD, FPF<5.0 μm measured and averaged over the duration of the inhalation.

Water content of the respirable dry powders comprising respirable dry particles can be measured by a Karl Fisher titration machine, or by a Thermogravimetric Analysis or Thermal Gravimetric Analysis (TGA). Karl Fischer titration uses coulometric or volumetric titration to determine trace amounts of water in a sample. TGA is a method of thermal analysis in which changes in weight of materials are measured as a function of temperature (with constant heating rate), or as a function of time (with constant temperature and/or constant mass loss). TGA may be used to determine the water content or residual solvent content of the material being tested.

The invention also relates to respirable dry powders comprising respirable dry particles produced using any of the methods described herein.

The respirable dry particles of the invention can also be characterized by the chemical, physical, aerosol, and solid-state stability of the therapeutic agents and excipients that the respirable dry particles comprise. The chemical stability of the constituent salts can affect important characteristics of the respirable particles including shelf-life, proper storage conditions, and acceptable environments for administration, biological compatibility, and effectiveness of the salts. Chemical stability can be assessed using techniques well known in the art. One example of a technique that can be used to assess chemical stability is reverse phase high performance liquid chromatography (RP-HPLC).

Methods for Preparing Acid Content Containing Dry Powders and Dry Particles

Respirable dry powders that comprise respirable dry particles can be formulated to include an acid content using suitable methods, for example, by adding a suitable acid, such as a strong acid, for example, hydrochloric acid, to an aqueous feedstock and spray drying the feedstock. A feedstock that contains the components of the dry powder can be produced using any suitable method. In one example, a tiotropium salt (such as tiotropium bromide, tiotropium chloride, or combinations thereof), amino acid (such as leucine), and any other desired excipients such as salt (such as sodium chloride) are added to a suitable solvent (e.g., water) and acid is then added to the mixture. The solution is stirred until it is clear to produce the feedstock. The amount of acid that is added is sufficient to decrease the growth of tiotropium impurities in the spray dried respirable dry powder over time. Generally, acid is added to achieve a desired molar ratio of 1) acid to amino acid (e.g., leucine) in the feedstock, or 2) acid to tiotropium salt in the feedstock, and correspondingly, in the respirable dry powder. Suitable molar ratios of 1) acid to amino acid (e.g., leucine) in the feed stock, and/or 2) acid to tiotropium salt in the feedstock, and correspondingly, in the respirable dry powder are described herein. In some preferred embodiments, the feed stock contains a molar ratio of acid to amino acid in the range of about 0.002 to about 1, and/or a molar ratio of acid to tiotropium in the range of about 2 to about 1000. The feedstock solution is then pumped into a spray dryer by means of a spray nozzle (such as a two-fluid nozzle). The nozzle atomizes the liquid feedstock into droplets that dry in the spray dryer to make respirable dry particles. These particles exit the spray dryer and proceed to a collector (such as a baghouse filter or a cyclone). The collected respirable dry powder comprising respirable dry particles is stored until being filled into receptacles for use in a dry powder inhaler (DPI) such as a capsule-based DPI, a blister-based DPI, or a reservoir-based DPI.

Measurement of the Chemical Integrity of the Tiotropium and its Impurities

The tiotropium content found in the respirable dry powders comprising respirable dry particles can be measured using a high-performance liquid chromatography (HPLC) system with an ultraviolet (UV) detector. The HPLC method was performed using an HPLC system with UV detection (HPLC-UV; Waters, Milford, Mass.) with Waters Xterra MS C18 column (5 μm, 3×100 mm; Waters, Milford, Mass.) to identify and quantify tiotropium in a range of 0.03 μg/mL to 1.27 μg/mL. The HPLC-UV system was set up with 100 μL injection volume, 40° C. column temperature, 240 nm detection wavelength, and isocratic elution with a mobile phase of 0.1% trifluoroacetic acid (Fisher Scientific, Pittsburgh, Pa.) and acetonitrile (Fisher Scientific, Pittsburgh, Pa.) (85:15) to determine tiotropium content in a 10 minute run time. Results are reported as both tiotropium and tiotropium bromide content.

Impurity testing of tiotropium containing respirable dry powders comprising respirable dry particles can be measured, for example, by two different methods of analysis. A reverse phase gradient HPLC method using a Zorbax, SB-C3 (150 mm×3.0 mm)3.5 μm column with UV detection at 240 nm is used for the detection of related substances A, B, C, E and F (described in Table 1) as outlined in Ph. Eur. Monograph 2420 Tiotropium Bromide Monohydrate. An LC-MS/MS gradient method utilizes a Waters HILIC (100 mm×4.6 mm) 3.0 μm column coupled with a quadrapole mass spectrometer to detect related substances G and H utilizing positive electrospray ionization and a transition of 170 to 94 m/z.

Therapeutic Use and Methods

The respirable dry powders comprising respirable dry particles of the present invention are suited for administration to the respiratory tract. The dry powders and dry particles of the invention can be administered to a subject in need thereof for the treatment of respiratory (e.g., pulmonary) diseases, such as chronic bronchitis, emphysema, chronic obstructive pulmonary disease, asthma, airway hyper responsiveness, seasonal allergic allergy, bronchiectasis, cystic fibrosis, pulmonary parenchymal inflammatory conditions and the like, and for the treatment, reduction in incidence or severity, and/or prevention of acute exacerbations of these chronic diseases, such as exacerbations caused by viral infections, bacterial infections, fungal infections or parasitic infections, or environmental allergens and irritants. In a preferred embodiment, the pulmonary disease is chronic bronchitis, emphysema, chronic obstructive pulmonary disease, or asthma. If desired, the respirable dry powders comprising respirable dry particles can be administered orally.

In a first aspect, the invention is a method for treating pulmonary diseases; in a second aspect, the invention is a method for the treatment, reduction in incidence or severity, or prevention of acute exacerbations; in a third aspect, the invention is a method for reducing inflammation; in a fourth aspect, the invention is a method for relieving symptoms; and, in a fifth aspect, the invention is a method for improving lung function; all of these aspect being targeted toward a patient with a respiratory disease and/or a chronic pulmonary disease. The diseases comprise chronic bronchitis, emphysema, chronic obstructive pulmonary disease, asthma, airway hyper responsiveness, seasonal allergic allergy, bronchiectasis, cystic fibrosis and the like, comprising administering to the respiratory tract of a subject in need thereof an effective amount of respirable dry particles or dry powder, as described herein. In a preferred embodiment, the pulmonary disease is chronic bronchitis, emphysema, chronic obstructive pulmonary disease, or asthma.

The respirable dry particles and dry powders can be administered to the respiratory tract of a subject in need thereof using any suitable method, such as instillation techniques, and/or an inhalation device, such as a dry powder inhaler (DPI) or metered dose inhaler (MDI). DPI configurations include: 1) Single-dose Capsule DPI, 2) Multi-dose Blister DPI, and 3) Multi-dose Reservoir DPI. Some representative capsule-based DPI units are RS-01 (Plastiape, Italy), Turbospin® (PH&T, Italy), Breezhaler® (Novartis, Switzerland), Aerolizer (Novartis, Switzerland), Podhaler® (Novartis, Switzerland), HandiHaler® (Boehringer Ingelheim, Germany), AIR® (Civitas, Massachusetts), Dose One® (Dose One, Maine), and Eclipse® (Rhone Poulenc Rorer). Spinhaler® (Fisons, Loughborough, U.K.), Rotahalers®, Diskhaler® and Diskus® (GlaxoSmithKline, Research Triangle Technology Park, North Carolina), FlowCaps® (Hovione, Loures, Portugal), Inhalators® (Boehringer-Ingelheim, Germany), Aerolizer® (Novartis, Switzerland). Some representative unit dose DPIs are Conix® (3M, Minnesota), Cricket® (Mannkind, California), Dreamboat® (Mannkind, California), Occoris® (Team Consulting, Cambridge, UK), Solis® (Sandoz), Trivair® (Trimel Biopharma, Canada), Twincaps® (Hovione, Loures, Portugal). Some representative blister-based DPI units are Diskus® (GlaxoSmithKline (GSK), UK), Diskhaler® (GSK), Taper Dry® (3M, Minnisota), Gemini® (GSK), Twincer® (University of Groningen, Netherlands), Aspirair® (Vectura, UK), Acu-Breathe® (Respirics, Minnisota, USA), Exubra® (Novartis, Switzerland), Gyrohaler® (Vectura, UK), Omnihaler® (Vectura, UK), Microdose® (Microdose Therapeutix, USA), Multihaler® (Cipla, India) Prohaler® (Aptar), Technohaler® (Vectura, UK), and Xcelovair® (Mylan, Pennsylvania). Some representative reservoir-based DPI units are Clickhaler® (Vectura), Next DPI® (Chiesi), Easyhaler® (Orion), Novolizer® (Meda), Pulmojet® (sanofi-aventis), Pulvinal® (Chiesi), Skyehaler® (Skyepharma), Duohaler® (Vectura), Taifun® (Akela), Flexhaler® (AstraZeneca, Sweden), Turbuhaler® (AstraZeneca, Sweden), and Twisthaler® (Merck), and others known to those skilled in the art.

Generally, inhalation devices (e.g., DPIs) are able to deliver a maximum amount of dry powder or dry particles in a single inhalation, which is related to the capacity of the blisters, capsules (e.g. size 000, 00, 0E, 0, 1, 2, 3, and 4, with respective volumetric capacities of 1.37 ml, 950 μl, 770 μl, 680 μl, 480 μl, 360 μl, 270 μl, and 200 μl) or other means that contain the dry particles or dry powders within the inhaler. Preferably, the blister has a volume of about 360 microliters or less, about 270 microliters or less, or more preferably, about 200 microliters or less, about 150 microliters or less, or about 100 microliters or less. Preferably, the capsule is a size 2 capsule, or a size 4 capsule. More preferably, the capsule is a size 3 capsule. Accordingly, delivery of a desired dose or effective amount may require two or more inhalations. Preferably, each dose that is administered to a subject in need thereof contains an effective amount of respirable dry particles or dry powder and is administered using no more than about four inhalations. For example, each dose of respirable dry particles or dry powder can be administered in a single inhalation or 2, 3, or 4 inhalations. The respirable dry particles and dry powders are preferably administered in a single, breath-activated step using a passive DPI. When this type of device is used, the energy of the subject's inhalation both disperses the respirable dry particles and draws them into the respiratory tract.

The respirable dry particles or dry powders can be preferably delivered by inhalation to a desired area within the respiratory tract, as desired. It is well-known that particles with an aerodynamic diameter (MMAD) of about 1 micron to about 3 microns, can be delivered to the deep lung. Larger MMAD, for example, from about 3 microns to about 5 microns can be delivered to the central and upper airways. Therefore, without wishing to be bound by theory, the invention has a MMAD of about 1 micron to about 5 microns, and preferentially, about 2.5 microns to about 4.5 microns, which preferentially deposits more of the therapeutic dose in the central airways than in the upper airways or in the deep lung.

For dry powder inhalers, oral cavity deposition is dominated by inertial impaction and so characterized by the aerosol's Stokes number (DeHaan et al. Journal of Aerosol Science, 35 (3), 309-331, 2003). For equivalent inhaler geometry, breathing pattern and oral cavity geometry, the Stokes number, and so the oral cavity deposition, is primarily affected by the aerodynamic size of the inhaled powder. Hence, factors which contribute to oral deposition of a powder include the size distribution of the individual particles and the dispersibility of the powder. If the MMAD of the individual particles is too large, e.g. above 5 μm, then an increasing percentage of powder will deposit in the oral cavity. Likewise, if a powder has poor dispersibility, it is an indication that the particles will leave the dry powder inhaler and enter the oral cavity as agglomerates. Agglomerated powder will perform aerodynamically like an individual particle as large as the agglomerate, therefore even if the individual particles are small (e.g., MMAD of 5 microns or less), the size distribution of the inhaled powder may have an MMAD of greater than 5 μm, leading to enhanced oral cavity deposition.

Therefore, it is desirable to have a powder in which the particles are small, dense, and dispersible such that the powders consistently deposit in the desired region of the respiratory tract. For example, the Respirable dry powders comprising respirable dry particles have a MMAD of about 5 microns or less, between about 1 micron and about 5 microns, preferably between about 2.5 microns and about 4.5 microns; are dense particles, for example have a high tap density and/or envelope density are desired, such as greater than 0.4 g/cm³, greater than 0.4 g/cm³ to about 1.2 g/cm³, about 0.45 g/cm³ or more, about 0.45 g/cm³ to about 1.2 g/cm³, about 0.5 g/cm³ or more, about 0.55 g/cm³ or more, about 0.55 g/cm³ to about 1.0 g/cm³, or about 0.6 g/cm³ to about 1.0 g/cm³; and are highly dispersible (e.g. 1/4 bar or alternatively, 0.5/4 bar of less than about 2.0, and preferably about 1.5 or less, or about 1.4 or less). The tap density and/or envelop density and MMAD are related theoretically to the VMGD by means of the following formula:

MMAD=VMGD*sqrt(envelope density or tap density).

If it is desired to deliver a high mass of therapeutic using a fixed volume dosing container, then, particles of higher tap density and/or envelope density are desired.

The respirable dry powders comprising respirable dry particles of the invention can be employed in compositions suitable for drug delivery via the respiratory system. For example, such compositions can include blends of the respirable dry particles of the invention and one or more other dry particles or powders, such as dry particles or powders that contain another active agent, or that consist of or consist essentially of one or more pharmaceutically acceptable excipients. The respirable dry particles can include blends of the dry particles with lactose, such as large lactose carrier particles that are greater than 10 microns, 20 microns to 500 microns, and preferably between 25 microns and 250 microns.

Respirable dry powders comprising respirable dry particles suitable for use in the methods of the invention can travel through the upper airways (i.e., the oropharynx and larynx), the lower airways, which include the trachea followed by bifurcations into the bronchi and bronchioli, and through the terminal bronchioli which in turn divide into respiratory bronchioli leading then to the ultimate respiratory zone, the alveoli or the deep lung. In one embodiment of the invention, most of the mass of respirable dry powders comprising respirable dry particles deposit in the deep lung. In another embodiment of the invention, delivery is primarily to the central airways. In another embodiment, delivery is to the upper airways. In a preferred embodiment, most of the mass of the respirable dry powders comprising respirable dry particles deposit in the conducting airways.

Suitable intervals between doses that provide the desired therapeutic effect can be determined based on the severity of the condition, overall well being of the subject and the subject's tolerance to respirable dry particles and dry powders and other considerations. Based on these and other considerations, a clinician can determine appropriate intervals between doses. Generally, respirable dry powders comprising respirable dry particles are administered once, twice or three times a day, as needed.

If desired or indicated, the respirable dry powders comprising respirable dry particles described herein can be administered with one or more other therapeutic agents. The other therapeutic agents can be administered by any suitable route, such as orally, parenterally (e.g., intravenous, intra-arterial, intramuscular, or subcutaneous injection), topically, by inhalation (e.g., intrabronchial, intranasal or oral inhalation, intranasal drops), rectally, vaginally, and the like. The respirable dry particles and dry powders can be administered before, substantially concurrently with, or subsequent to administration of the other therapeutic agent. Preferably, the respirable dry particles and dry powders and the other therapeutic agent are administered so as to provide substantial overlap of their pharmacologic activities.

In another aspect of the invention, the dry powders comprising dry particles of the present invention are suitable for administration to a patient via the mouth or the nose, or intravenously after reconstituting the dry powder into a physiologically acceptable solvent. For administration via the mouth, the liquid formulation may be aerosolized and inhaled through the mouth in order to be delivered to deeper parts of the respiratory tract of a patient. Alternatively, the dry powder comprising dry particles may be administered directly to the mouth as a powder, spray or aerosol. For administration via to the nose, the dry powder comprising dry particles may be administered directly to the nose as a powder, spray or aerosol for either local delivery or delivery to other parts of the respiratory tract of a patient. For administration intravenously, the powder may be reconstituted in a physiologically acceptable solvent. It may then be administered by any of the methods known in the art, for example, by intravenous injection.

### Liquid Formulations

Liquid formulations encompass a liquid containing one or more therapeutic agents such as tiotropium and one or more excipients, as a solution, suspension, emulsion or slurry. A liquid formulation may be a pharmaceutical liquid formulation that is suitable for administration to a patient in need of the therapeutic agent present in the formulation such as tiotropium. A liquid formulation may also be a feedstock liquid formulation that is suitable to be fed into a process that removes the liquid in order to form dry particles such as respirable dry particles such as by spray drying.

The liquid formulations contain tiotropium as an active ingredient. While any form of tiotropium may be used, preferred tiotropium salts added to a liquid to make the liquid formulation include tiotropium bromide, tiotropium chloride, and combinations thereof. The liquid formulations may also contain an additional therapeutic with tiotropium including corticosteroids, such as inhaled corticosteroids (ICS), long-acting beta agonists (LABA), short-acting beta agonists (SABA), anti-inflammatory agents, bifunctional muscarinic antagonist-beta2 agonist (MABA), and any combination thereof. The liquid formulations contain an amino acid. The amino acid can be, for example, leucine orglycine. The liquid formulations may optionally contain another excipient such as a salt, a carbohydrate, a sugar alcohol, and the like. The salt can be a sodium salt, a magnesium salt, a calcium salt, or the like. For example, the salt can be sodium chloride. The carbohydrate can be, for example, maltodextrin or lactose. The sugar alcohol can be, for example, mannitol. The liquid formulations also contain an acid, such as a strong acid. Some examples include hydrochloric acid, hydrobromic acid, nitric acid, and sulfuric acid. The components in the liquid formulation may be in any percentage provided that the described molar ratios are maintained. However, the following are examples of weight percentages of the components in the liquid formulation, on a solute or dry basis: the tiotropium salt may be about 0.01% to about 0.5%, the amino acid may be about 5% to about 40%, the optional sodium salt, such as sodium chloride, may be about 50% to about 90%, the optional one or more additional therapeutic agents may be up to about 30%, where all percentages are weight percentages on a dry basis and all the components of the liquid formulation amount to 100%.

Ranges of the components in the liquid formulations, when presented on a solute basis, are as follows. The molar ratio of acid to amino acid is about 0.0005 to about 5.0, about 0.001 to about 2.0, about 0.002 to about 1, about 0.005 to about 0.5, about 0.01 to about 0.1, or about 0.1 to about 0.5. In a preferred embodiment, the ratio is about 0.002 to about 1. The molar ratio of acid to tiotropium in liquid formulations is about 0.5 to about 2000, about 1.0 to about 1000, about 2 to about 1000, about 5 to about 500, about 10 to about 250, about 25 to about 100, or about 100 to about 250. In a preferred embodiment, the ratio is about 2 to about 1000. Alternatively or in addition, the acid is added in sufficient quantities to product, for example, the pH of the feedstock is between 2.0 and 5.0, between 2.5 and 4.0, or between 3 and 3.5.

When the liquid formulations are pharmaceutical liquid formulations they may be administered to a patient to the mouth (e.g., buccal administration, mouthwash, mouth rinse, etc.) or nose (e.g., nasal administration, nasal wash, nasal rinse, paranasal sinuses, etc.), or via the mouth or nose to the respiratory tract such as to you upper airways (e.g., pharynx, larynx) or to the deep airways (e.g., trachea, bronchi, bronchioles, and alveoli). Alternatively, the pharmaceutical liquid formulations may be administered intravenously. Aerosolization may be achieved, for example, with a pressurized metered dose inhaler, a mist or soft-mist inhaler, or a nebulizer. For administration via the mouth or nose, the liquid formulation may be sprayed or aerosolized for either local delivery or delivery to the other parts of the respiratory tract of a patient. Aerosolization to the mouth may be achieved, for example, with a pressurized metered dose inhaler, a mist or soft-mist inhaler, or a nebulizer. Aerosolization to the nose may be achieved via a nasal spray, a mist or soft-mist inhaler, a nebulizer or a pressurized metered dose inhaler. For administration intravenously, any of the methods known in the art may be utilized, for example, by injection or infusion. The concentration of the tiotropium in the liquid formulations may be different for the different modes of administration. However, generally the tiotropium may have a concentration in the liquid formulations of about 0.0002% to about 0.2%, or about 0.002% to about 0.02%.

Liquid formulations may be characterized based on standard measurement techniques and parameters such as those found in U.S. Pat. No. 8,387,895 and US 20120067343, which are herein incorporated by reference.

The pharmaceutical liquid formulations may be packaged and/or stored at a temperature of about 15° C. to about 30° C. The liquid formulation may be packaged as is ordinary in the field. The tiotropium purity and/or impurities can be measured during storage, e.g., 1 week after packaging, 2 weeks after packaging, 3 weeks after packaging, 1 month after packaging, 2 months after packaging, 3 months after packaging, 6 months after packaging, 9 months after packaging, 12 months after packaging, 18 months after packaging, or 24 months after packaging. During storage, the purity of each therapeutic agent is 96.0% or greater, the total amount of Impurities A, B, C, E, F, G and H is 2.0% or less, and/or Impurity A and Impurity B are each 1.0% or less.

A feedstock liquid formulation can be produced using any suitable method. In one example, tiotropium (such as a tiotropium salt, e.g., tiotropium bromide, tiotropium chloride, or combinations thereof), an amino acid (such as leucine), and optionally, any other desired excipients such as a salt (such as a sodium salt, e.g., sodium chloride) are added to a suitable solvent (e.g., water) or solvents (e.g., water and an organic solvent). Acid may be added subsequently to the mixture and together with the other solutes. The solution is stirred until it is clear to produce the feedstock. The amount of acid that is added is sufficient to decrease the growth of tiotropium impurities in the spray dried respirable dry powder over time. Generally, acid is added to achieve a desired molar ratio of 1) acid to amino acid (e.g., leucine) in the feedstock, or 2) acid to tiotropium salt in the feedstock, and correspondingly, in the respirable dry powder. Suitable molar ratios of 1) acid to amino acid (e.g., leucine) in the feed stock, and/or 2) acid to tiotropium salt in the feedstock, and correspondingly, in the respirable dry powder are described herein. For example, the feed stock contains a molar ratio of acid to amino acid in the range of about 0.002 to about 1, and/or a molar ratio of acid to tiotropium in the range of about 2 to about 1000. The feedstock solution is then pumped into a spray dryer by means of a spray nozzle (such as a two-fluid nozzle). The nozzle atomizes the liquid feedstock into droplets that dry in the spray dryer to make respirable dry particles.

When the liquid formulation is a feedstock liquid formulation, acid may be added to the feedstock formulation to increase the chemical stability of the tiotropium. For example, when acid is added, the feedstock formulation may be maintained for a period of time before manufacturing to make the dry powder and dry particles. The feedstock can be made up to 1 day, 2 days, 3 days, 4 days, 5 days, 6 days, or 7 days before manufacturing and still remain stable when stored between 15 to 25 degree Celsius; or up to 1 week, 2 weeks, 3 weeks, 4 weeks, 2 months or 3 months before manufacturing and still remain stable when stored under refrigerated conditions.

### EXEMPLIFICATION

Materials used in the following Examples and their sources are listed below. Sodium chloride, and L-leucine were obtained from Sigma-Aldrich Co. (St. Louis, Mo.), Spectrum Chemicals (Gardena, Calif.), or Merck (Darmstadt, Germany) Tiotropium bromide was obtained from RIA International (East Hanover, N.J.) or Teva API (Tel Aviv, Israel). Ultrapure (Type II ASTM) water was from a water purification system (Millipore Corp., Billerica, Mass.), or equivalent.

Methods:

Tiotropium Content/Purity Using HPLC.

Tiotropium content was measured using a high-performance liquid chromatography (HPLC) system with an ultraviolet (UV) detector. The HPLC method was performed using an HPLC system with UV detection (HPLC-UV; Waters, Milford, Mass.) with Waters Xterra MS C18 column (5 μm, 3 mm×100 mm; Waters, Milford, Mass.) to identify and quantify tiotropium in a range of 0.03 μg/mL to 1.27 μg/mL. The HPLC-UV system was set up with 100 μL injection volume, 40° C. column temperature, 240 nm detection wavelength, and isocratic elution with a mobile phase of 0.1% trifluoroacetic acid (Fisher Scientific, Pittsburgh, Pa.) and acetonitrile (Fisher Scientific, Pittsburgh, Pa.) (85:15) to determine tiotropium content in a 10 minute run time. Results are reported as both tiotropium and tiotropium bromide content.

Impurities Test.

Testing of tiotropium containing Respirable dry powders comprising respirable dry particles can be measured by two different methods of analysis. A reverse phase gradient HPLC method using a Zorbax, SB-C3(150 mm×3.0 mm)3.5 μm column with UV detection at 240 nm is used for the detection of related substances A, B, C, E and F (described in Table 1) as outlined in Ph. Eur. Monograph 2420 Tiotropium Bromide Monohydrate. An LC-MS/MS gradient method utilizes a Waters HILIC (100 mm×4.6 mm) 3.0 μm column coupled with a quadrapole mass spectrometer to detect related substances G and H utilizing positive electrospray ionization and a transition of 170 to 94 m/z.

Geometric or Volume Diameter.

Volume median diameter (x50 or Dv50), which may also be referred to as volume median geometric diameter (VMGD), was determined using a laser diffraction technique. The equipment consisted of a HELOS diffractometer and a RODOS dry powder disperser (Sympatec, Inc., Princeton, N.J.). The RODOS disperser applies a shear force to a sample of particles, controlled by the regulator pressure (typically set at 1.0 bar with maximum orifice ring pressure) of the incoming compressed dry air. The pressure settings may be varied to vary the amount of energy used to disperse the powder. For example, the dispersion energy may be modulated by changing the regulator pressure from 0.2 bar to 4.0 bar. Powder sample is dispensed from a microspatula into the RODOS funnel. The dispersed particles travel through a laser beam where the resulting diffracted light pattern produced is collected, typically using an R1 lens, by a series of detectors. The ensemble diffraction pattern is then translated into a volume-based particle size distribution using the Fraunhofer diffraction model, on the basis that smaller particles diffract light at larger angles. Using this method, geometric standard deviation (GSD) for the volume diameter was also determined.

Volume median diameter can also be measured using a method where the powder is emitted from a dry powder inhaler device. The equipment consisted of a Spraytec laser diffraction particle size system (Malvern, Worcestershire, UK), “Spraytec”. Powder formulations were filled into size 3 HPMC capsules (Capsugel V-Caps) by hand with the fill weight measured gravimetrically using an analytical balance (Mettler Tolerdo XS205). A capsule based passive dry powder inhaler (RS01 Model 7, High resistance Plastiape S.p.A.) was used which had a specific resistance of 0.036 kPa^{1/2}LPM⁻¹. Flow rate and inhaled volume were set using a timer controlled solenoid valve with flow control valve (TPK2000, Copley Scientific). Capsules were placed in the dry powder inhaler, punctured and the inhaler sealed inside a cylinder. The cylinder was connected to a positive pressure air source with steady air flow through the system measured with a mass flow meter and its duration controlled with a timer controlled solenoid valve. The exit of the dry powder inhaler was exposed to room pressure and the resulting aerosol jet passed through the laser of the diffraction particle sizer (Spraytec) in its open bench configuration before being captured by a vacuum extractor. The steady air flow rate through the system was initiated using the solenoid valve and the particle size distribution was measured via the Spraytec at 1 kHz for the duration of the single inhalation maneuver with a minimum of 2 seconds. Particle size distribution parameters calculated included the volume median diameter (Dv50) and the geometric standard deviation (GSD) and the fine particle fraction (FPF) of particles less than 5 micrometers in diameter. At the completion of the inhalation duration, the dry powder inhaler was opened, the capsule removed and re-weighed to calculate the mass of powder that had been emitted from the capsule during the inhalation duration (capsule emitted powder mass or CEPM).

Fine Particle Fraction.

The aerodynamic properties of the powders dispersed from an inhaler device were assessed with an Mk-II 1 ACFM Andersen Cascade Impactor (Copley Scientific Limited, Nottingham, UK) (ACI) or a Next Generation Impactor (Copley Scientific Limited, Nottingham, UK) (NGI). The ACI instrument was run in controlled environmental conditions of 18 to 25° C. and relative humidity (RH) between 25 and 35%. The instrument consists of eight stages that separate aerosol particles based on inertial impaction. At each stage, the aerosol stream passes through a set of nozzles and impinges on a corresponding impaction plate. Particles having small enough inertia will continue with the aerosol stream to the next stage, while the remaining particles will impact upon the plate. At each successive stage, the aerosol passes through nozzles at a higher velocity and aerodynamically smaller particles are collected on the plate. After the aerosol passes through the final stage, a filter collects the smallest particles that remain, called the “final collection filter”. Gravimetric and/or chemical analyses can then be performed to determine the particle size distribution. A short stack cascade impactor, also referred to as a collapsed cascade impactor, is also utilized to allow for reduced labor time to evaluate two aerodynamic particle size cut-points. With this collapsed cascade impactor, stages are eliminated except those required to establish fine and coarse particle fractions.

The impaction techniques utilized allowed for the collection of two or eight separate powder fractions. The capsules (HPMC, Size 3; Capsugel Vcaps, Peapack, N.J.) were hand filled with powder to a specific weight and placed in a hand-held, breath-activated dry powder inhaler (DPI) device, the high resistance RS01 DPI or the ultra high resistance UHR2 DPI (both by Plastiape, Osnago, Italy). The capsule was punctured and the powder was drawn through the cascade impactor operated at a flow rate of 60.0 L/min for 2.0 s. At this flowrate, the calibrated cut-off diameters for the eight stages are 8.6, 6.5, 4.4, 3.3, 2.0, 1.1, 0.5 and 0.3 microns and for the two stages used with the short stack cascade impactor, based on the Andersen Cascade Impactor, the cut-off diameters are 5.6 microns and 3.4 microns. The fractions were collected by placing filters in the apparatus and determining the amount of powder that impinged on them by gravimetric measurements or chemical measurements on an HPLC. The fine particle fraction of the total dose of powder (FPF_{TD}) less than or equal to an effective cut-off aerodynamic diameter was calculated by dividing the powder mass recovered from the desired stages of the impactor by the total particle mass in the capsule. Results are reported for the eight-stage normal stack cascade impactor as the fine particle fraction of less than 4.4 microns (FPF T_{D}<4.4 microns) and the fine particle fraction of less than 2.0 microns (FPF_{TD}<2.0 microns), and the two-stage short stack cascade impactor as the fine particle fraction of less than 5.6 microns (FPF T_{D}<5.6 microns) and the fine particle fraction of less than 3.4 microns (FPF_{TD}<3.4 microns). The fine particle fraction can alternatively be calculated relative to the recovered or emitted dose of powder by dividing the powder mass recovered from the desired stages of the impactor by the total powder mass recovered in the impactor.

Similarly, for FPF measurements utilizing the NGI, the NGI instrument was run in controlled environmental conditions of 18 to 25° C. and relative humidity (RH) between 25 and 35%. The instrument consists of seven stages that separate aerosol particles based on inertial impaction and can be operated at a variety of air flow rates. At each stage, the aerosol stream passes through a set of nozzles and impinges on a corresponding impaction surface. Particles having small enough inertia will continue with the aerosol stream to the next stage, while the remaining particles will impact upon the surface. At each successive stage, the aerosol passes through nozzles at a higher velocity and aerodynamically smaller particles are collected on the plate. After the aerosol passes through the final stage, a micro-orifice collector collects the smallest particles that remain. Chemical analyses can then be performed to determine the particle size distribution. The capsules (HPMC, Size 3; Capsugel Vcaps, Peapack, N.J.) were hand filled with powder to a specific weight and placed in a hand-held, breath-activated dry powder inhaler (DPI) device, the high resistance RS01 DPI or the ultra high resistance RS01 DPI (both by Plastiape, Osnago, Italy). The capsule was punctured and the powder was drawn through the cascade impactor operated at a specified flow rate for 2.0 Liters of inhaled air. At the specified flow rate, the cut-off diameters for the stages were calculated. The fractions were collected by placing wetted filters in the apparatus and determining the amount of powder that impinged on them by chemical measurements on an HPLC. The fine particle fraction of the total dose of powder (FPF_{TD}) less than or equal to an effective cut-off aerodynamic diameter was calculated by dividing the powder mass recovered from the desired stages of the impactor by the total particle mass in the capsule. Results are reported for the NGI as the fine particle fraction of less than 5.0 microns (FPF T_{D}<5.0 microns)

Aerodynamic Diameter.

Mass median aerodynamic diameter (MMAD) was determined using the information obtained by the Andersen Cascade Impactor (ACI). The cumulative mass under the stage cut-off diameter is calculated for each stage and normalized by the recovered dose of powder. The MMAD of the powder is then calculated by linear interpolation of the stage cut-off diameters that bracket the 50th percentile. An alternative method of measuring the MMAD is with the Next Generation Pharmaceutical Impactor (NGI). Like the ACI, the MMAD is calculated with the cumulative mass under the stage cut-off diameter is calculated for each stage and normalized by the recovered dose of powder. The MMAD of the powder is then calculated by linear interpolation of the stage cut-off diameters that bracket the 50th percentile.

Fine Particle Dose.

The fine particle dose (FPD) is determined using the information obtained from the ACI. Alternatively, the FPD is determined using the information obtained from the NGI. The fine particle dose indicates the mass of one or more therapeutics in a specific size range and can be used to predict the mass which will reach a certain region in the respiratory tract. The fine particle dose can be measured gravimetrically or chemically. If measured gravimetrically, since the dry particles are assumed to be homogenous, the mass of the powder on each stage and collection filter can be multiplied by the fraction of therapeutic agent in the formulation to determine the mass of therapeutic. If measured chemically, the powder from each stage or filter is collected, separated, and assayed for example on an HPLC to determine the content of the therapeutic. The cumulative mass deposited on the final collection filter, and stages 6, 5, 4, 3, and 2 for a single dose of powder, contained in one or more capsules, actuated into the ACI is equal to the fine particle dose less than 4.4 microns (FPD<4.4 microns). The cumulative mass deposited on the final collection filter, and stages 6, 5 and 4 for a single dose of powder, contained in one or more capsules, actuated into the ACI is equal to the fine particle dose less than 2.0 microns (FPD<2.0 microns). The quotient of these two values is expressed as FPD<2.0 μm/FPD<4.4 μm. Other ratios measured were: FPD<2.0 μm/FPD<5.0 μm and FPD<3.0 μm/FPD<5.0 μm. The higher the ratio, the higher the percentage of therapeutic that enters the lungs is expected to penetrate to the alveolar regions of the lung. The lower the ratio, the lower the percentage of therapeutic that enters the lungs is expected to penetrate to the alveolar regions of the lung. For some therapies that target the central or conducting airways, a lower ratio such as less than 40%, less than 30%, or less than 20% is desired. For other therapies that target the deep lung, a higher ratio such as 40% or greater, 50% or greater, or 60% or greater is desired. Similarly, for FPD measurements utilizing the NGI, the NGI instrument was run as described in the Fine Particle Fraction description in the Exemplification section. The cumulative mass deposited on each of the stages at the specified flow rate is calculated and the cumulative mass corresponding to a 5.0 micrometer diameter particle is interpolated. This cumulative mass for a single dose of powder, contained in one or more capsules, actuated into the NGI is equal to the fine particle dose less than 5.0 microns (FPD<5.0 microns).

Emitted Geometric or Volume Diameter.

The volume median diameter (Dv50) of the powder after it is emitted from a dry powder inhaler, which may also be referred to as volume median geometric diameter (VMGD), was determined using a laser diffraction technique via the Spraytec diffractometer (Malvern, Inc.). Powder was filled into size 3 capsules (V-Caps, Capsugel) and placed in a capsule based dry powder inhaler (RS01 Model 7 High resistance, Plastiape, Italy), or DPI, and the DPI sealed inside a cylinder. The cylinder was connected to a positive pressure air source with steady air flow through the system measured with a mass flow meter and its duration controlled with a timer controlled solenoid valve. The exit of the dry powder inhaler was exposed to room pressure and the resulting aerosol jet passed through the laser of the diffraction particle sizer (Spraytec) in its open bench configuration before being captured by a vacuum extractor. The steady air flow rate through the system was initiated using the solenoid valve. A steady air flow rate was drawn through the DPI typically at 60 L/min for a set duration, typically of 2 seconds. Alternatively, the air flow rate drawn through the DPI was sometimes run at 15 L/min, 20 L/min, or 30 L/min. The resulting geometric particle size distribution of the aerosol was calculated from the software based on the measured scatter pattern on the photodetectors with samples typically taken at 1000 Hz for the duration of the inhalation. The Dv50, GSD, FPF<5.0 μm measured were then averaged over the duration of the inhalation.

The Emitted Dose (ED) refers to the mass of therapeutic which exits a suitable inhaler device after a firing or dispersion event. The ED is determined using a method based on USP Section 601 Aerosols, Metered-Dose Inhalers and Dry Powder Inhalers, Delivered-Dose Uniformity, Sampling the Delivered Dose from Dry Powder Inhalers, United States Pharmacopeia convention, Rockville, Md., 13th Revision, 222-225, 2007. Contents of capsules are dispersed using either the RS01 HR inhaler at a pressure drop of 4 kPa and a typical flow rate of 60 LPM or the UHR2 RS01 at a pressure drop of 4 kPa and a typical flow rate of 39 LPM. The emitted powder is collected on a filter in a filter holder sampling apparatus. The sampling apparatus is rinsed with a suitable solvent such as water and analyzed using an HPLC method. For gravimetric analysis a shorter length filter holder sampling apparatus is used to reduce deposition in the apparatus and the filter is weighed before and after to determine the mass of powder delivered from the DPI to the filter. The emitted dose of therapeutic is then calculated based on the content of therapeutic in the delivered powder. Emitted dose can be reported as the mass of therapeutic delivered from the DPI or as a percentage of the filled dose.

Capsule Emitted Powder Mass.

A measure of the emission properties of the powders was determined by using the information obtained from the Andersen Cascade Impactor tests or emitted geometric diameter by Spraytec. The filled capsule weight was recorded at the beginning of the run and the final capsule weight was recorded after the completion of the run. The difference in weight represented the amount of powder emitted from the capsule (CEPM or capsule emitted powder mass). The CEPM was reported as a mass of powder or as a percent by dividing the amount of powder emitted from the capsule by the total initial particle mass in the capsule. While the standard CEPM was measured at 60 L/min, it was also measured at 15 L/min, 20 L/min, or 30 L/min.

Tap Density.

Tap density was measured using a modified method requiring smaller powder quantities, following USP<616> with the substitution of a 1.5 cc microcentrifuge tube (Eppendorf AG, Hamburg, Germany) or a 0.3 cc section of a disposable serological polystyrene micropipette (Grenier Bio-One, Monroe, N.C.) with polyethylene caps (Kimble Chase, Vineland, N.J.) to cap both ends and hold the powder. Instruments for measuring tap density, known to those skilled in the art, include but are not limited to the Dual Platform Microprocessor Controlled Tap Density Tester (Vankel, Cary, N.C.) or a SOTAX Tap Density Tester model TD2 (Horsham, Pa.). Tap density is a standard, approximated measure of the envelope mass density. The envelope mass density of an isotropic particle is defined as the mass of the particle divided by the minimum spherical envelope volume within which it can be enclosed.

Bulk Density.

Bulk density was estimated prior to tap density measurement procedure by dividing the weight of the powder by the unconsolidated volume of the powder, as estimated using the volumetric measuring device.

Thermogravimetric Analysis.

Thermogravimetric analysis (TGA) was performed using a Thermogravimetric Analyzer Q500 (TA Instruments, New Castle, Del.). The samples were placed into an open aluminum DSC pan with the tare weight previously recorded by the instrument. The following method was employed: Ramp 10.00° C./min from ambient (−35° C.). to 200° C. The weight loss was reported as a function of temperature up to 150° C. TGA allows for the calculation of the water content of the dry powder.

Liquid Feedstock Preparation for Spray Drying.

Spray drying homogenous particles requires that the ingredients of interest be solubilized in solution or suspended in a uniform and stable suspension. Sodium chloride, leucine and tiotropium bromide are sufficiently water-soluble to prepare suitable spray drying solutions. Alternatively, ethanol or another organic solvent can be used.

Spray Drying Using Niro Spray Dryer.

Dry powders were produced by spray drying utilizing a Niro Mobile Minor spray dryer (GEA Process Engineering Inc., Columbia, Md.) with powder collection from a cyclone, a product filter or both. Atomization of the liquid feed was performed using a co-current two-fluid nozzle either from Niro (GEA Process Engineering Inc., Columbia, Md.) or a Spraying Systems (Carol Stream, Ill.) 1/4 J two-fluid nozzle with gas cap 67147 and fluid cap 2850SS, although other two-fluid nozzle setups are also possible. In some embodiments, the two-fluid nozzle can be in an internal mixing setup or an external mixing setup. Additional atomization techniques include rotary atomization or a pressure nozzle. The liquid feed was fed using gear pumps (Cole-Parmer Instrument Company, Vernon Hills, Ill.) directly into the two-fluid nozzle or into a static mixer (Charles Ross & Son Company, Hauppauge, N.Y.) immediately before introduction into the two-fluid nozzle. An additional liquid feed technique includes feeding from a pressurized vessel. Nitrogen or air may be used as the drying gas, provided that moisture in the air is at least partially removed before its use. Pressurized nitrogen or air can be used as the atomization gas feed to the two-fluid nozzle. The drying gas inlet temperature can range from 70° C. to 300° C. and outlet temperature from 30° C. to 120° C. with a liquid feedstock rate of 10 mL/min to 100 mL/min. The gas supplying the two-fluid atomizer can vary depending on nozzle selection and for the Niro co-current two-fluid nozzle can range from 5 kg/hr to 50 kg/hr or for the Spraying Systems 1/4J two-fluid nozzle can range from 30 g/min to 150 g/min. The atomization gas rate can be set to achieve a certain gas to liquid mass ratio, which directly affects the droplet size created. The pressure inside the drying drum can range from +3 ″WC to −6 ″WC. Spray dried powders can be collected in a container at the outlet of the cyclone, onto a cartridge or baghouse filter, or from both a cyclone and a cartridge or baghouse filter.

Spray Drying Using Büchi Spray Dryer.

Dry powders were prepared by spray drying on a Büchi B-290 Mini Spray Dryer (BUCHI Labortechnik A G, Flawil, Switzerland) with powder collection from either a standard or High Performance cyclone. The system was run either with air or nitrogen as the drying and atomization gas in open-loop (single pass) mode. When run using air, the system used the Büchi B-296 dehumidifier to ensure stable temperature and humidity of the air used to spray dry. Furthermore, when the relative humidity in the room exceeded 30% RH, an external LG dehumidifier (model 49007903, LG Electronics, Englewood Cliffs, N.J.) was run constantly. When run using nitrogen, a pressurized source of nitrogen was used. Furthermore, the aspirator of the system was adjusted to maintain the system pressure at −2.0″ water column Atomization of the liquid feed utilized a Büchi two-fluid nozzle with a 1.5 mm diameter or a Schlick 970-0 atomizer with a 0.5 mm liquid insert (Düsen-Schlick GmbH, Coburg, Germany) Inlet temperature of the process gas can range from 100° C. to 220° C. and outlet temperature from 30° C. to 120° C. with a liquid feedstock flowrate of 3 mL/min to 10 mL/min. The two-fluid atomizing gas ranges from 25 mm to 45 mm (300 LPH to 530 LPH) for the Büchi two-fluid nozzle and for the Schlick atomizer an atomizing air pressure of upwards of 0.3 bar. The aspirator rate ranges from 50% to 100%.

Spray Drying Using ProCepT Formatrix.

Dry powders were prepared by spray drying on a ProCepT Formatrix R&D spray dryer (ProCepT nv, Zelzate, Belgium). The system was run in open loop configuration using room air in a manufacturing suite controlled to <60% RH. The drying gas flow rate can range from 0.2 to 0.5 m³/min. The bi-fluid nozzle was equipped for atomization with liquid tips from 0.15-1.2 mm. The atomization gas pressure could vary from about 0.5 bar to 6 bar. The system was equipped with either the small or medium cyclone. The inlet temperature of the spray dryer can range from about 100° C. to 190° C., with an outlet temperature from about 40° C. to about 95° C. The liquid feedstock flowrate can range from about 0.1 to 15 mL/min. Process parameters were controlled via the ProCepT human-machine interface (HMI) and all parameters were recorded electronically.

### Example 1. Two-Component Formulations that Support that Leucine is Likely the Cause of the Formation of Impurity B (N-Demethyl Tiotropium)

Excipient compatibility with tiotropium was assessed by evaluating two-component spray dried formulations (i.e., tiotropium with either sodium chloride or leucine) where the tiotropium was amorphous, the sodium chloride was crystalline, and the leucine was partially crystalline and partially amorphous, as well as physical mixtures (i.e. powder blends) of crystalline tiotropium with either crystalline sodium chloride or crystalline leucine. The chemical stability of these formulations was measured at various time points during storage.

### A: Powder Preparation

The feedstock solutions were spray dried in order to make dry particles. For Formulation I, the liquid feedstock was batch mixed, the total solids concentration was 30 g/L, the amount of tiotropium bromide in solution was 0.3 g/L, the amount of L-leucine in the solution was 29.7 g/L and the final aqueous feedstock was clear. L-leucine was the form of leucine used in this example. For Formulation II, the liquid feedstock was batch mixed, the total solids concentration was 30 g/L, the amount of tiotropium bromide in solution was 0.3 g/L, the amount of sodium chloride in the solution was 29.7 g/L and the final feedstock was mixed until it was clear.

Dry powders of Formulations I and II were manufactured from these feedstocks by spray drying on the Büchi B-290 Mini Spray Dryer (BUCHI Labortechnik A G, Flawil, Switzerland) with high performance cyclone powder collection. The system was run in open-loop (single pass) mode using nitrogen as the drying and atomization gas. Atomization of the liquid feed utilized a 1.5 mm nozzle cap. The aspirator of the system was adjusted to maintain the system pressure at −2.0″ water column

The following spray drying conditions were followed to manufacture the dry powders. For Formulations I and II, the liquid feedstock solids concentration was 30 g/L, the process gas inlet temperature was 180° C., the process gas outlet temperature was 80° C., the drying gas flowrate was 18.0 kg/hr, the atomization gas flowrate was 20.0 g/min, and the liquid feedstock flowrate was 6.0 mL/min. The resulting dry powder formulations are reported in Table 2.

The physical mixtures were made as follows. For Formulation III, the material was geometrically mixed by way of adding 0.990 grams of L-leucine to 0.010 grams of tiotropium bromide followed by 10 minutes of mechanical blending. For Formulation IV, the material was geometrically mixed by way of adding 3.600 grams of sodium chloride to 0.400 grams of tiotropium bromide followed by 10 minutes of mechanical blending. The resulting physical mixtures are reported in Table 2.

TABLE 2
Composition of Formulations I-IV

| | | Solids Composition (w/w) | | |
|---|---|---|---|---|
| | | Tiotropium | | Sodium |
| | Manufacturing | bromide | L-leucine | chloride |
| Formulation | Condition | (%) | (%) | (%) |
| I | Spray Dried | 1.0% | 99.0% | 0.0% |
| II | Spray Dried | 1.0% | 0.0% | 99.0% |
| III | Physical Mixture | 1.0% | 99.0% | 0.0% |
| IV | Physical Mixture | 1.0% | 0.0% | 99.0% |

B. Powder Characterization

The chemical stability of Formulations I, II, III and IV was assessed by measuring the tiotropium purity and Impurity B amounts using HPLC. The measurements were made after storing the formulations for 1) 24 hours at 80° C. less than 10% RH, 2) for 0.5 months at 40° C. stored in an open dish at 60% RH and for 0.5 months at 40° C. stored packaged at 75% RH, and 3) for 1.5 months at 40° C. stored in an open dish at 60% RH and for 0.5 months at 40° C. stored packaged at 75% RH.

For Formulation I, the tiotropium was spray dried with L-leucine. The tiotropium was fully amorphous and the L-leucine was present in both crystalline form and amorphous. Formulation I exhibited a rise in Impurity B and thereby a drop in tiotropium purity at the stress conditions of 80° C. Formulation I exhibited a slight rise in Impurity B at 0.5 months, 40° C., and stored packaged at 75% RH. This rise in Impurity B became more prominent at the 1.5 month time point. Formulations II, III and IV did not show any significant signs of increase of Impurity B nor in the reduction in tiotropium purity at any condition. Results indicated that tiotropium was more likely to be prone to degradation when in amorphous form and spray dried with leucine than in any other combination tested. Results for the measurement of Impurity B are found in Table 3. Results for the measurement of tiotropium purity are found in Table 4.

TABLE 3
Impurity B Levels during Stability

| | Formula- | Formula- | Formula- | Formula- |
|---|---|---|---|---|
| | tion 1 | tion 2 | tion 3 | tion 4 |
| T = 0 hours | 0.00 | 0.00 | 0.00 | 0.00 |
| T = 24 hours; 80° C., | 13.17 | 0.00 | 0.00 | 0.00 |
| stored packaged at 0% | | | | |
| RH | | | | |
| T = 0.5 months; 40° C., | 0.00 | 0.00 | 0.00 | 0.00 |
| stored open to 60% RH | | | | |
| T = 0.5 months; 40° C., | 0.41 | 0.00 | 0.00 | 0.00 |
| stored packaged at 75% | | | | |
| RH | | | | |
| T = 1.5 months; 40° C., | 0.07 | 0.00 | 0.01 | 0.00 |
| stored open to 60% RH | | | | |
| T = 1.5 months; 40° C., | 1.08 | 0.00 | 0.00 | 0.00 |
| stored packaged at 75% | | | | |
| RH | | | | |

TABLE 4
Tiotropium Purity during Stability

| | Formula- | Formula- | Formula- | Formula- |
|---|---|---|---|---|
| | tion 1 | tion 2 | tion 3 | tion 4 |
| T = 0 hours | 99.73 | 99.87 | 99.88 | 99.86 |
| T = 24 hours; 80° C., | 86.18 | 99.87 | 99.85 | 99.88 |
| stored packaged | | | | |
| at <10% RH | | | | |
| T = 0.5 months; 40° C., | 99.69 | 99.73 | 99.88 | 99.87 |
| stored open to 60% RH | | | | |
| T = 0.5 months; 40° C., | 99.28 | 99.87 | 99.87 | 99.89 |
| stored packaged at 75% | | | | |
| RH | | | | |
| T = 1.5 months; 40° C., | 99.58 | 99.86 | 99.81 | 99.86 |
| stored open to 60% RH | | | | |
| T = 1.5 months; 40° C., | 99.59 | 99.71 | 99.83 | 99.89 |
| stored packaged at 75% | | | | |
| RH | | | | |

### Example 2. Acid Containing Formulations with Varied Acid Contents

### A. Powder Preparation

Feedstock solutions were prepared and used to manufacture dry powders comprising neat, dry particles containing tiotropium bromide, sodium chloride, L-leucine, and varying amounts of hydrochloric acid (HCl). L-leucine was the form of leucine used in this example. Table 5 lists the components of the feedstock formulations used in preparation of the dry powders comprised of dry particles.

TABLE 5
Feedstock compositions

| | | Feedstock Composition (w/w) | | | | |
|---|---|---|---|---|---|---|
| | | | Tiotropium | Sodium | L- | Hydro- |
| | Feedstock | Water | bromide | chloride | leucine | chloric |
| Formulation | pH | (%) | (%) | (%) | (%) | acid (%) |
| V | 2.0 | 97.06 | 0.002 | 2.337 | 0.586 | 0.158 |
| VI | 3.0 | 97.02 | 0.002 | 2.336 | 0.586 | 0.037 |
| VII | 4.0 | 97.02 | 0.002 | 2.336 | 0.586 | 0.004 |
| VIII | 5.0 | 97.07 | 0.002 | 2.337 | 0.586 | 0.0004 |

The feedstock solutions that were used to spray dry particles were made as follows. For Formulation V, the liquid feedstock was batch mixed, the total solids concentration was 31.76 g/L, the amount of tiotropium bromide in solution was 0.02 g/L, the amount of sodium chloride in the solution was 24.07 g/L, the amount of L-leucine in the solution was 6.04 g/L, the amount of hydrochloric acid in the solution was 1.63 g/L and the final aqueous feedstock was clear. For Formulation VI, the liquid feedstock was batch mixed, the total solids concentration was 30.51 g/L, the amount of tiotropium bromide in solution was 0.02 g/L, the amount of sodium chloride in the solution was 24.07 g/L, the amount of L-leucine in the solution was 6.04 g/L, the amount of hydrochloric acid in the solution was 0.38 g/L and the final feedstock was clear. For Formulation VII, the liquid feedstock was batch mixed, the total solids concentration was 30.18 g/L, the amount of tiotropium bromide in solution was 0.02 g/L, the amount of sodium chloride in the solution was 24.07 g/L, the amount of L-leucine in the solution was 6.04 g/L, the amount of hydrochloric acid in the solution was 0.04 g/L and the final feedstock was clear. For Formulation VIII, the liquid feedstock was batch mixed, the total solids concentration was 30.13 g/L, the amount of tiotropium bromide in solution was 0.02 g/L, the amount of sodium chloride in the solution was 24.07 g/L, the amount of L-leucine in the solution was 6.02 g/L, the amount of hydrochloric acid in the solution was 0.004 g/L and the final feedstock was clear. Feedstock volumes were 0.375 L, which supported manufacturing campaigns of one hour.

Dry powders of Formulations V through VIII were manufactured from these feedstocks by spray drying on the Büchi B-290 Mini Spray Dryer (BUCHI Labortechnik A G, Flawil, Switzerland) with cyclone powder collection. The system was run in open-loop (single pass) mode using nitrogen as the drying and atomization gas. Atomization of the liquid feed utilized a Schlick 970-0 atomizer with a 0.5 mm liquid insert. The aspirator of the system was adjusted to maintain the system pressure at −2.0″ water column.

The following spray drying conditions were followed to manufacture the dry powders. For Formulations V and VIII, the liquid feedstock solids concentration was approximately 30 g/L, the process gas inlet temperature was 185° C., the process gas outlet temperature was 77° C., the drying gas flowrate was 18.0 kg/hr, the atomization gas flowrate was 1.824 kg/hr, the atomization gas backpressure at the atomizer inlet was 36 psig and the liquid feedstock flowrate was 6.0 mL/min. The resulting dry powder formulations are reported in Table 6.

TABLE 6
Dry powder compositions, dry basis

| | Composition (w/w) | | | |
|---|---|---|---|---|
| | Tiotropium | Sodium | | Hydrochloric |
| Formu- | bromide | chloride | L-leucine | acid |
| lation | (%) | (%) | (%) | (%) |
| V | 0.067 | 75.794 | 19.007 | 5.133 |
| VI | 0.069 | 78.908 | 19.788 | 1.236 |
| VII | 0.07 | 79.777 | 20.006 | 0.148 |
| VIII | 0.07 | 79.887 | 20.032 | 0.013 |
| | Acid:Leucine | Acid:Leucine | Acid:Tio | Acid:Tio |
| Formu- | Ratio | Ratio | Ratio | Ratio |
| lation | (mol/mol) | (wt/wt) | (mol/mol) | (wt/wt) |
| V | 0.971 | 0.270 | 991.5 | 76.6 |
| VI | 0.225 | 0.062 | 231.8 | 17.9 |
| VII | 0.027 | 0.007 | 27.4 | 2.1 |
| VIII | 0.002 | 0.001 | 2.4 | 0.2 |

### B. Powder Characterization

The dry powder physical and aerosol properties of Formulations V-VIII were assessed. Properties assessed were tapped density, mass median aerodynamic diameter (MMAD) and fine particles doses (FPD) as found using all eight stages of the Anderson Cascade Impactor (ACI), and volumetric median geometric diameter (microns) and 1 bar to 4 bar (1/4 bar) ratio as found using the RODOS HELOS laser diffraction unit. Results are shown in Table 7. The results show that the tapped densities were all greater than 0.5 g/cc, the MMAD were all between 2.8 and 3.4 microns, the FPD (<4.4 microns) were all between 3.7 and 4.6 micrograms, the FPD (<2.0 microns) were varied, ranging from 0.238 micrograms to 1.792 micrograms, resulting in varied FPD (<2.0 microns)/FPD (<4.4 microns) ratios of 0.06 to 0.39. The VMGD were all between 2.1 and 2.5, with the 1/4 bar ratios all below 1.2.

TABLE 7
Dry powder physical and aerosol properties

| Formulation | V | VI | VII | VIII | Method |
|---|---|---|---|---|---|
| Tapped density (g/cc) | 0.61 | 0.54 | 0.66 | 0.76 | SOTAX TD1 |
| MMAD (microns) | 3.36 | 3.20 | 2.81 | 3.35 | ACI8 |
| FPD < 4.4 microns | 3.65 | 4.00 | 4.62 | 3.95 | ACI8 |
| FPD < 2.0 microns | 0.959 | 0.238 | 1.792 | 1.008 | ACI8 |
| FPD < 2.0 microns/ | 0.26 | 0.06 | 0.39 | 0.26 | ACI8 |
| FPD < 4.4 microns | | | | | |
| VMGD (microns) | 2.06 | 2.50 | 2.23 | 2.32 | RODOS |
| ¼ bar ratio | 1.03 | 1.17 | 1.14 | 1.19 | RODOS |

The chemical stability of Formulations V-VIII was assessed at 80° C. The powders were sealed in amber glass vials in an environmentally controlled chamber set to 10% RH. The formation of the known degradants Impurity A and Impurity B were monitored over 24 hours (24 h) and 72 hours (72h). The results are shown in Tables 8-10. In comparison to Formulation VIII, which only contained 0.013 wt % acid, Formulations V-VII, which each had a higher loading of acid than 0.013 wt %, each showed a reduction in the formation of Impurities A and B over time. This indicated that more acid in the spray dried powder contributed positively to reducing Impurity A and B formation over time.

TABLE 8
Tiotropium Purity During Stability for Varied Acid
Content Formulations: 80° C. Stability Data

| | Formula- | Formula- | Formula- | Formula- |
|---|---|---|---|---|
| | tion V | tion VI | tion VII | tion VIII |
| T = 0 hours | 100.00 | 100.00 | 100.00 | 100.00 |
| T = 24 hours; 80° C., | 97.26 | 97.63 | 97.07 | 84.95 |
| 10% RH | | | | |
| T = 72 hours; 80° C., | 94.02 | 85.47 | 88.33 | 44.21 |
| 10% RH | | | | |

TABLE 9
Impurity A Levels Reported as Percent During Stability for
Varied Acid Content Formulations: 80° C. Stability Data

| | Formula- | Formula- | Formula- | Formula- |
|---|---|---|---|---|
| | tion V | tion VI | tion VII | tion VIII |
| T = 0 hours | 0.00 | 0.00 | 0.00 | 0.00 |
| T = 24 hours; 80° C., | 0.38 | 0.33 | 0.80 | 1.11 |
| 10% RH | | | | |
| T = 72 hours; 80° C., | 0.69 | 0.59 | 0.44 | 3.64 |
| 10% RH | | | | |

TABLE 10
Impurity B Levels during Stability for Varied Acid
Content Formulations: 80° C. Stability Data

| | Formula- | Formula- | Formula- | Formula- |
|---|---|---|---|---|
| | tion V | tion VI | tion VII | tion VIII |
| T = 0 hours | 0.00 | 0.00 | 0.00 | 0.00 |
| T = 24 hours; 80° C., | 0.00 | 1.68 | 1.71 | 10.35 |
| 10% RH | | | | |
| T = 72 hours; 80° C., | 0.67 | 12.98 | 10.57 | 45.91 |
| 10% RH | | | | |

The chemical stability of Formulations V-VIII was assessed at 40° C. under packaged 75% relative humidity (RH) conditions. The packaged samples were sealed in amber glass vials in an environmentally controlled chamber set to 10% RH. The formation of the known degradants Impurity A and Impurity B were monitored. The results are shown in Tables 11 and 12.

TABLE 11
Varied Acid Content Formulations: Impurity A

| | Formula- | Formula- | Formula- | Formula- |
|---|---|---|---|---|
| | tion V | tion VI | tion VII | tion VIII |
| T = 0 hours | 0.00 | 0.00 | 0.00 | 0.00 |
| T = 2 weeks; Packaged | 0.00 | 0.00 | 0.25 | 0.00 |
| (40° C., 75% RH) | | | | |
| T = 6 weeks; Packaged | 0.17 | 0.00 | 0.28 | 0.21 |
| (40° C., 75% RH) | | | | |
| T = 2 weeks; Open | 1.05 | 0.00 | 0.41 | 0.00 |
| (40° C., 60% RH) | | | | |
| T = 6 weeks; Open | 1.99 | 0.25 | 0.16 | 0.62 |
| (40° C., 60% RH) | | | | |

TABLE 12
Varied Acid Content Formulations: Impurity B

| | Formula- | Formula- | Formula- | Formula- |
|---|---|---|---|---|
| | tion V | tion VI | tion VII | tion VIII |
| T = 0 hours | 0.00 | 0.00 | 0.00 | 0.00 |
| T = 2 weeks; Packaged | 0.00 | 0.00 | 0.00 | 0.28 |
| (40° C., 75% RH) | | | | |
| T = 6 weeks; Packaged | 0.00 | 0.23 | 0.23 | 0.00 |
| (40° C., 75% RH) | | | | |
| T = 2 weeks; Open | 0.00 | 0.00 | 0.00 | 0.00 |
| (40° C., 60% RH) | | | | |
| T = 6 weeks; Open | 0.00 | 0.00 | 0.00 | 0.85 |
| (40° C., 60% RH) | | | | |

The results indicate that levels of Impurity B were reduced at acid content levels above 0.013 wt %. Impurity A levels varied, but were highest for the highest acid content, Formulation V. Comparison of the degradation profiles of both Impurities A and B would indicate that a minimum level of acid content is required to realize the advantage, but that there may be diminishing returns at excess levels, thus suggesting the existence of an optimal level for maximum benefit.

### Example 3. Acid Containing Formulations with Varied Acid and L-Leucine Contents

### A. Powder Preparation

Feedstock solutions were prepared and used to manufacture dry powders comprising neat, dry particles containing tiotropium bromide, sodium chloride, and varying amounts of L-leucine, and hydrochloric acid. Powders were prepared in duplicate. Table 13 lists the components of the feedstock formulations used in preparation of the dry powders comprised of dry particles.

TABLE 13
Feedstock compositions

| | Feedstock Composition (w/w) | | | | |
|---|---|---|---|---|---|
| | | Tiotropium | Sodium | | Hydrochloric |
| | Water | bromide | chloride | L-leucine | acid |
| Formulation | (%) | (%) | (%) | (%) | (%) |
| IX | 97.08 | 0.002 | 2.32 | 0.58 | 0.0160 |
| X | 97.08 | 0.002 | 2.33 | 0.58 | 0.0018 |
| XI | 97.08 | 0.002 | 1.72 | 1.17 | 0.0320 |
| XII | 97.08 | 0.002 | 1.75 | 1.17 | 0.0035 |

The feedstock solutions that were used to spray dry particles were made as follows. For Formulation IX, the liquid feedstock was batch mixed, the total solids concentration was 30 g/L, the amount of tiotropium bromide in solution was 0.021 g/L, the amount of sodium chloride in the solution was 23.82 g/L, the amount of leucine in the solution was 6.0 g/L, the amount of hydrochloric acid in the solution was 0.16 g/L, and the final aqueous feedstock was clear. For Formulation X, the liquid feedstock was batch mixed, the total solids concentration was 30 g/L, the amount of tiotropium bromide in solution was 0.021 g/L, the amount of sodium chloride in the solution was 23.96 g/L, the amount of leucine in the solution was 6.0 g/L, the amount of hydrochloric acid in the solution was 0.018 g/L, and the final feedstock was clear. For Formulation XI, the liquid feedstock was batch mixed, the total solids concentration was 30 g/L, the amount of tiotropium bromide in solution was 0.021 g/L, the amount of sodium chloride in the solution was 17.65 g/L, the amount of leucine in the solution was 6.0 g/L, the amount of hydrochloric acid in the solution was 0.33 g/L, and the final feedstock was clear. For Formulation XII, the liquid feedstock was batch mixed, the total solids concentration was 30 g/L, the amount of tiotropium bromide in solution was 0.021 g/L, the amount of sodium chloride in the solution was 17.95 g/L, the amount of leucine in the solution was 6.0 g/L, the amount of hydrochloric acid in the solution was 0.036 g/L, and the final feedstock was clear. Feedstock volumes were 0.55 L, which supported manufacturing campaigns of 1.5 hours.

Dry powders of Formulations IX through XII were manufactured from these feedstocks by spray drying on the Büchi B-290 Mini Spray Dryer (BUCHI Labortechnik A G, Flawil, Switzerland) with cyclone powder collection. The system was run in open-loop (single pass) mode using nitrogen as the drying and atomization gas. Atomization of the liquid feed utilized a Schlick 970-0 atomizer with a 0.5 mm liquid insert. The aspirator of the system was adjusted to maintain the system pressure at −2.0″ water column.

The following spray drying conditions were followed to manufacture the dry powders. For Formulations IX-XII, the liquid feedstock solids concentration was 30 g/L, the process gas inlet temperature was 174° C., the process gas outlet temperature was 77° C., the drying gas flowrate was 18.0 kg/hr, the atomization gas flowrate was 1.824 kg/hr, the atomization gas backpressure at the atomizer inlet was 36 psig and the liquid feedstock flowrate was 6.0 mL/min. The resulting dry powder formulations are reported in Table 14.

TABLE 14
Dry powder compositions, dry basis

| | Composition (w/w) | | | |
|---|---|---|---|---|
| | Tiotropium | Sodium | | Hydrochloric |
| Formu- | bromide | chloride | L-leucine | acid |
| lation | (%) | (%) | (%) | (%) |
| IX | 0.07 | 79.404 | 19.986 | 0.54 |
| X | 0.07 | 79.874 | 19.986 | 0.07 |
| XI | 0.07 | 58.868 | 39.972 | 1.09 |
| XII | 0.07 | 59.828 | 39.972 | 0.13 |
| | Acid:Leucine | Acid:Leucine | Acid:Tio | Acid:Tio |
| Formu- | Ratio | Ratio | Ratio | Ratio |
| lation | (mol/mol) | (wt/wt) | (mol/mol) | (wt/wt) |
| IX | 0.10 | 0.027 | 99.8 | 7.7 |
| X | 0.01 | 0.004 | 12.9 | 1.0 |
| XI | 0.10 | 0.027 | 201.5 | 15.6 |
| XII | 0.01 | 0.003 | 24.0 | 1.9 |

### B. Powder Characterization

The dry powder physical and aerosol properties of Formulation IX-XII were assessed. Properties assessed were tapped density, mass median aerodynamic diameter (MMAD) and fine particles doses (FPD) as found using all eight stages of the Anderson Cascade Impactor (ACI), and volumetric median geometric diameter (microns) and 1 bar to 4 bar (1/4 bar) ratio as found using the RODOS HELOS laser diffraction unit. Results are shown in Table 15. The results show that the tapped densities of Formulations IX-XI were greater than 0.4 g/cc, the MMAD were all between 2.5 and 3.5 microns, the FPD (<4.4 microns) were all between 3.6 and 4.5 micrograms, the FPD (<2.0 microns) were all between 1.0 micrograms to 2.0 micrograms, resulting in FPD (<2.0 microns)/FPD (<4.4 microns) ratios of 0.27 to 0.45. The VMGD were all between 1.9 and 2.6, with the 1/4 bar ratios all below 1.4.

TABLE 15
Dry powder physical and aerosol properties

| Formulation | IX | X | XI | XII | Method |
|---|---|---|---|---|---|
| Tapped density (g/cc) | 0.46 | 0.52 | 0.43 | 0.31 | SOTAX TD1 |
| MMAD (μm) | 2.98 | 3.01 | 2.48 | 3.46 | ACI8 |
| FPD < 4.4 microns | 4.46 | 4.45 | 4.42 | 3.57 | ACI8 |
| FPD < 2.0 microns | 1.38 | 1.36 | 2.00 | 0.98 | ACI8 |
| FPD < 2.0 μm/ | 0.31 | 0.31 | 0.45 | 0.27 | ACI8 |
| FPD < 4.4 μm | | | | | |
| VMGD (μm) | 1.96 | 1.94 | 2.44 | 2.59 | RODOS/HELOS |
| 1:4 bar ratio | 1.33 | 1.26 | 1.18 | 1.33 | RODOS/HELOS |

The chemical stability of Formulations IX-XII was assessed at 80° C. The powders were sealed in amber glass vials in an environmentally controlled chamber set to 10% RH. The formation of the known degradants Impurity A and Impurity B were monitored over 24h and 72h. The results are shown in Tables 16& 17 and are reported as averages of samples prepared from duplicate formulations.

TABLE 16
Varied Acid Content Formulations -
Impurity A: 80° C. Stability Data

| | Formula- | Formula- | Formula- | Formula- |
|---|---|---|---|---|
| | tion IX | tion X | tion XI | tion XII |
| T = 0 hours | 0.00 | 0.00 | 0.00 | 0.00 |
| T = 24 hours; Packaged | 0.00 | 0.00 | 0.00 | 0.00 |
| (80° C., 10% RH) | | | | |
| T = 72 hours; Packaged | 0.14 | 0.16 | 0.00 | 0.18 |
| (80° C., 10% RH) | | | | |

TABLE 17
Varied Acid Content Formulations -
Impurity B: 80° C. Stability Data

| | Formula- | Formula- | Formula- | Formula- |
|---|---|---|---|---|
| | tion IX | tion X | tion XI | tion XII |
| T = 0 hours | 0.00 | 0.00 | 0.00 | 0.00 |
| T = 24 hours; Packaged | 0.62 | 2.72 | 1.29 | 2.17 |
| (80° C., 10% RH) | | | | |
| T = 72 hours; Packaged | 1.72 | 7.03 | 1.63 | 5.17 |
| (80° C., 10% RH) | | | | |

Minimal levels of Impurity A were observed in all formulations after 72h storage at 80° C. The formulations with increased levels of acid content showed decreased levels of Impurity B, regardless of leucine level over 72h at 80° C.

The chemical stability of Formulations IX-XII was assessed at 40° C. under open-dish 60% RH conditions. The open dish samples were prepared by transferring powders to amber glass scintillation vials and affixing a single-ply task wipe over the mouth of the vial to avoid ingress of foreign materials while still allowing access to the environment. The formation of the known degradants Impurity A and Impurity B were monitored. The results are shown in Tables 18 and 19 and are reported as averages of samples prepared from duplicate formulations. The results from the 40° C./60% RH open dish stability study indicate no significant growth of Impurity B occurs for any level of acid content or leucine content. No strong correlation between Impurity A and acid content or leucine content was observed.

TABLE 18
Varied Acid Content Formulations- Impurity
A: 40° C./60% RH Open Dish Stability Data

| | Formula- | Formula- | Formula- | Formula- |
|---|---|---|---|---|
| | tion IX | tion X | tion XI | tion XII |
| T = 0 hours | 0.00 | 0.00 | 0.00 | 0.00 |
| T = 2 weeks; Open | 0.24 | 0.38 | 0.39 | 0.25 |
| (40° C., 60% RH) | | | | |
| T = 1 month; Open | 0.47 | 0.38 | 0.82 | 0.72 |
| (40° C., 60% RH) | | | | |
| T = 3 months; Open | 0.52 | 0.32 | 2.28 | 1.29 |
| (40° C., 60% RH) | | | | |

TABLE 19
Varied Acid Content Formulations- Impurity
B: 40° C./60% RH Open Dish Stability Data

| | Formula- | Formula- | Formula- | Formula- |
|---|---|---|---|---|
| | tion IX | tion X | tion XI | tion XII |
| T = 0 hours | 0.00 | 0.00 | 0.00 | 0.00 |
| T = 2 weeks; Open | 0.00 | 0.00 | 0.00 | 0.00 |
| (40° C., 60% RH) | | | | |
| T = 1 month; Open | 0.00 | 0.00 | 0.00 | 0.00 |
| (40° C., 60% RH) | | | | |
| T = 3 months; Open | 0.93 | 0.00 | 0.00 | 0.00 |
| (40° C., 60% RH) | | | | |

The chemical stability of Formulations IX-XII was assessed at 40° C. packaged at 75% RH. The packaged samples were sealed in amber glass vials in an environmentally controlled chamber set to 10% RH. The formation of the known degradants Impurity A and Impurity B were monitored. The results are shown in Tables 20 and 21 and are reported as averages of samples prepared from duplicate formulations.

TABLE 20
Varied Acid Content Formulations - Impurity
A: 40° C./75% RH Packaged Stability Data

| | Formula- | Formula- | Formula- | Formula- |
|---|---|---|---|---|
| | tion IX | tion X | tion XI | tion XII |
| T = 0 hours | 0.00 | 0.00 | 0.00 | 0.00 |
| T = 2 weeks; Packaged | 0.00 | 0.13 | 0.00 | 0.00 |
| (40° C., 75% RH) | | | | |
| T = 1 month; Packaged | 0.00 | 0.16 | 0.00 | 0.17 |
| (40° C., 75% RH) | | | | |
| T = 3 months; Packaged | 0.18 | 0.46 | 0.25 | 0.50 |
| (40° C., 75% RH) | | | | |

TABLE 21
Varied Acid Content Formulations - Impurity
B: 40° C./75% RH Packaged Stability Data

| | Formula- | Formula- | Formula- | Formula- |
|---|---|---|---|---|
| | tion IX | tion X | tion XI | tion XII |
| T = 0 hours | 0.00 | 0.00 | 0.00 | 0.00 |
| T = 2 weeks; Packaged | 0.00 | 0.29 | 0.00 | 0.35 |
| (40° C., 75% RH) | | | | |
| T = 1 month; Packaged | 0.25 | 0.56 | 0.27 | 0.55 |
| (40° C., 75% RH) | | | | |
| T = 3 months; Packaged | 0.34 | 0.76 | 0.68 | 1.54 |
| (40° C., 75% RH) | | | | |

The results from the 40° C./75% RH packaged storage show that a relatively increased level of acid is able to reduce levels of Impurity A and Impurity B in formulations with a range of leucine levels from 20 to 40 wt %.

### Example 4. Conservation of Acid Content from Feedstock to Respirable Dry Powder

Feedstock solutions were prepared in water and were spray dried to manufacture respirable dry powders comprising respirable dry particles containing tiotropium bromide, L-leucine, and varying amounts of hydrochloric acid (HCl). Table 22 below lists the components of the feedstock formulations used in preparation of the dry powders comprised of dry particles. Weight percentages are given on a dry basis. Adjustment of pH by way of hydrochloric acid addition was made after the addition and solubilization of tiotropium bromide and L-leucine.

### A: Powder Preparation

The feedstock solutions that were used to spray dry particles were made as follows. For Formulation XIII-XVI, the liquid feedstock was batch mixed, the total solids concentration was 20 g/L, the amount of tiotropium bromide in solution was 0.2 g/L, the amount of leucine in the solution was 19.8 g/L and the final aqueous feedstock was clear. The feedstock was split into four equal parts. Each of the four volume was adjusted by HCl addition to the target pH listed in Table 22 below.

Dry powders of Formulations XIII-XVII were manufactured from these feedstocks by spray drying on the Büchi B-290 Mini Spray Dryer (BUCHI Labortechnik A G, Flawil, Switzerland) with high performance cyclone powder collection. The system was run in open-loop (single pass) mode using nitrogen as the drying and atomization gas. Atomization of the liquid feed utilized a 1.5 mm nozzle cap. The aspirator of the system was adjusted to maintain the system pressure at −2.0″ water column

The following spray drying conditions were followed to manufacture the dry powders. For Formulations XIII-XVI, the liquid feedstock solids concentration was 30 g/L, the process gas inlet temperature was 170° C., the process gas outlet temperature was 80° C., the drying gas flowrate was 18.0 kg/hr, the atomization gas flowrate was 20.0 g/min, and the liquid feedstock flowrate was 6.0 mL/min. The resulting dry powder formulations are reported in Table 22.

TABLE 22
Feedstock compositions

| | Feedstock pH | | |
|---|---|---|---|
| | (adjusted by | Solids Composition (w/w) | |
| | hydrochloric | Tiotropium bromide | L-leucine |
| Formulation | acid) | (%) | (%) |
| XIII | 2.0 | 1.0% | 99.0% |
| XIV | 3.0 | 1.0% | 99.0% |
| XV | 4.0 | 1.0% | 99.0% |
| XVI | 5.0 | 1.0% | 99.0% |

The acid content in the spray dried powders was shown to remain intact and no significant loss of acid was observed due to the spray drying manufacturing process. The observation was made by way of reconstitution of the powders at concentrations identical to the solution feedstock and comparing the initial versus post spray drying pH value. Table 23 shows the pH of the initial feedstock solutions versus the aqueous solutions of reconstituted powders at an equivalent solids loading of 20 g/L. The pH in the reconstituted solution is near equivalent to the initial feedstock indicating that no HCl was lost during the spray drying process.

TABLE 23
pH Measurements of Feedstock

| | Feedstock | | Reconstituted | |
|---|---|---|---|---|
| | Solid | pH of | Feedstock Solid | pH of |
| Formu- | Concentration | Initial | Concentration | Reconstituted |
| lation | (g/L) | Feedstock | (g/L) | Solution |
| XIII | 20 | 2.03 | 20 | 2.01 |
| XIV | 20 | 3.00 | 20 | 2.95 |
| XV | 20 | 4.02 | 20 | 4.06 |
| XVI | 20 | 5.01 | 20 | 5.10 |

### Example 5. Acid Containing Formulations with Varied Acid and L-Leucine Contents

### A. Powder Preparation

Feedstock solutions were prepared and used to manufacture dry powders comprising neat, dry particles containing tiotropium bromide, sodium chloride, L-leucine, and varying amounts of hydrochloric acid. Table 24 lists the components of the feedstock formulations used in preparation of the dry powders comprised of dry particles.

TABLE 24
Feedstock compositions

| | Feedstock Composition (w/w) | | | | |
|---|---|---|---|---|---|
| | | Tiotropium | Sodium | | Hydrochloric |
| | Water | bromide | chloride | L-leucine | acid |
| Formulation | (%) | (%) | (%) | (%) | (%) |
| IX | 97.08 | 0.002 | 2.32 | 0.58 | 0.0160 |
| X | 97.08 | 0.002 | 2.33 | 0.58 | 0.0018 |

The feedstock solutions that were used to spray dry particles were made as follows. For Formulation IX, the liquid feedstock was batch mixed, the total solids concentration was 30 g/L, the amount of tiotropium bromide in solution was 0.021 g/L, the amount of sodium chloride in the solution was 23.82 g/L, the amount of leucine in the solution was 6.0 g/L, the amount of hydrochloric acid in the solution was 0.16 g/L, and the final feedstock was clear. For Formulation X, the liquid feedstock was batch mixed, the total solids concentration was 30 g/L, the amount of tiotropium bromide in solution was 0.021 g/L, the amount of sodium chloride in the solution was 23.96 g/L, the amount of leucine in the solution was 6.0 g/L, the amount of hydrochloric acid in the solution was 0.018 g/L, and the final feedstock was clear. Feedstock volumes were 2.4 L, which supported manufacturing campaigns of 1.0 hours.

Dry powders of Formulations IX and X were manufactured from these feedstocks by spray drying on the GEA Niro Mobil Minor Spray Dryer (MFR) with cyclone powder collection. The system was run in open-loop (single pass) mode using nitrogen as the drying and atomization gas. Atomization of the liquid feed utilized a Niro 2-fluid atomizer with a 1.0 mm cap and 2.5 mm separator. The aspirator of the system was adjusted to maintain the system pressure at −2.0″ water column

The following spray drying conditions were followed to manufacture the dry powders. For Formulations IX and X, the liquid feedstock solids concentration was 30 g/L, the process gas inlet temperature was 180° C., the process gas outlet temperature was 77° C., the drying gas flowrate was 80.0 kg/hr, the atomization gas flowrate was 1.260 kg/hr, the atomization gas backpressure at the atomizer inlet was 23.0 psig and the liquid feedstock flowrate was 40 mL/min. The resulting dry powder formulations are reported in Table 25.

TABLE 25
Dry powder compositions, dry basis

| | Composition (w/w) | | | |
|---|---|---|---|---|
| | Tiotropium | Sodium | | Hydrochloric |
| Formu- | bromide | chloride | L-leucine | acid |
| lation | (%) | (%) | (%) | (%) |
| IX | 0.07 | 79.404 | 19.986 | 0.54 |
| X | 0.07 | 79.874 | 19.986 | 0.07 |
| | Acid:Leucine | Acid:Leucine | Acid:Tio | Acid:Tio |
| Formu- | Ratio | Ratio | Ratio | Ratio |
| lation | (mol/mol) | (wt/wt) | (mol/mol) | (wt/wt) |
| IX | 0.10 | 0.027 | 99.8 | 7.7 |
| X | 0.01 | 0.004 | 12.9 | 1.0 |

### B. Powder Characterization and Physicochemical Stability

The dry powder physical, chemical and aerosol properties of Formulation IX and X were assessed at 2-8° C., 25° C./60% RH, and 40 C°/75% RH packaged storage conditions. Properties assessed were mass median aerodynamic diameter (MMAD) and fine particles doses (FPD) as found using all stages of the Next Generation Impactor (NGI), volumetric median geometric diameter (microns) and 1 bar to 4 bar (1/4 bar) ratio as found using the RODOS HELOS laser diffraction unit. Results for aerodynamic and volumetric particle size are shown in Table 26 & 27, respectively. The results show that the MMAD were all between 2.98 and 3.62 microns, the FPD (<5.0 microns) were all between 1.32 and 1.84 micrograms, the FPD (<2.0 microns) were all between 0.30 micrograms to 0.65 micrograms, resulting in FPD (<2.0 microns)/FPD (<5.0 microns) ratios of 0.23 to 0.36. The VMGD were all between 1.86 and 2.32, with the 1/4 bar ratios all below 1.39.

TABLE 26
Dry powder aerosol performance stability

| | | | | | | | | FPD(<2.0 | |
|---|---|---|---|---|---|---|---|---|---|
| | | | | | | | | microns)/FPD(<5.0 | |
| Storage | Time | MMAD (μm) | | FPD < 5 μm | | FPD < 2 μm | | microns) ratio | |
| Condition | (months) | IX | X | IX | X | IX | X | IX | X |
| Time zero | 0 | 3.62 | 3.13 | 1.62 | 1.78 | 0.45 | 0.63 | 0.28 | 0.35 |
| 2-8° C., | 1 | N.T. | N.T. | N.T. | N.T. | N.T. | N.T. | N.T. | N.T. |
| packaged | 4 | N.T. | N.T. | N.T. | N.T. | N.T. | N.T. | N.T. | N.T. |
| | 6 | 3.52 | 2.99 | 1.61 | 1.84 | 0.39 | 0.65 | 0.24 | 0.35 |
| 25° C./60% RH, | 1 | 3.44 | 2.98 | 1.63 | 1.82 | 0.46 | 0.64 | 0.28 | 0.35 |
| packaged | 4 | 3.47 | 2.98 | 1.52 | 1.79 | 0.43 | 0.65 | 0.28 | 0.36 |
| | 6 | 3.48 | 3.00 | 1.55 | 1.79 | 0.41 | 0.60 | 0.27 | 0.34 |
| 40° C./75% RH, | 1 | 3.53 | — | 1.55 | — | 0.42 | — | 0.27 | — |
| packaged | 4 | 3.59 | 3.05 | 1.37 | 1.63 | 0.35 | 0.54 | 0.26 | 0.33 |
| | 6 | 3.62 | 3.19 | 1.32 | 1.55 | 0.30 | 0.45 | 0.23 | 0.29 |

N.T. means that the time point/condition was not tested.

TABLE 27
Dry powder physical performace stability

| | | | |
|---|---|---|---|
| Storage | Time | VMGD (μm) | ¼ bar ratio |

| Condition | (months) | IX | X | IX | X |
|---|---|---|---|---|---|
| N/A | 0 | 2.10 | 1.91 | 1.30 | 1.26 |
| 2-8° C., | 1 | 2.18 | 1.86 | 1.39 | 1.23 |
| packaged | 4 | 2.20 | 1.93 | 1.38 | 1.27 |
| | 6 | 2.32 | 2.07 | 1.23 | 1.16 |

The chemical stability of Formulations IX and X was assessed. The bulk powders were sealed in HDPE bottles in an environmentally controlled chamber set to 10% RH. The bulk capsules were sealed in HDPE bottles in at 30% RH. Both capsule and bulk powder samples were sealed in foil pouches. The formation of the known degradants Impurity A and Impurity B were monitored over 1 month, 4 months, and 6 months. The results are shown in Tables 28 & 29 and are reported as averages of samples prepared from duplicate formulations.

TABLE 28
Varied Acid Content Formulations -
Impurity A: 6 month Stability Data

| Storage | Time | Formulation IX | | Formulation X | |
|---|---|---|---|---|---|
| Condition | (months) | Powder | Capsule | Powder | Capsule |
| N/A | 0 | 0.09 | 0.00 | 0.10 | 0.00 |
| 2-8° C., | 1 | 0.00 | — | 0.00 | — |
| packaged | 4 | 0.00 | — | 0.11 | — |
| | 6 | 0.09 | 0.00 | 0.11 | 0.00 |
| 25° C./60% RH, | 1 | — | 0.00 | — | 0.00 |
| packaged | 4 | — | 0.17 | — | 0.15 |
| | 6 | — | 0.16 | — | 0.17 |
| 40° C./75% RH, | 1 | — | 0.12 | — | — |
| packaged | 4 | — | 0.49 | — | 0.04 |
| | 6 | — | 0.46 | — | 0.52 |

TABLE 29
Varied Acid Content Formulations -
Impurity B: 6 month Stability Data

| Storage | Time | Formulation IX | | Formulation X | |
|---|---|---|---|---|---|
| Condition | (months) | Powder | Capsule | Powder | Capsule |
| N/A | 0 | 0.00 | 0.00 | 0.00 | 0.00 |
| 2-8° C., | 1 | 0.00 | — | 0.00 | — |
| packaged | 4 | 0.00 | — | 0.00 | — |
| | 6 | 0.00 | 0.00 | 0.00 | 0.00 |
| 25° C./60% RH, | 1 | — | 0.00 | — | 0.00 |
| packaged | 4 | — | 0.21 | — | 0.35 |
| | 6 | — | 0.26 | — | 0.46 |
| 40° C./75% RH, | 1 | — | 0.47 | — | — |
| packaged | 4 | — | 1.37 | — | 2.76 |
| | 6 | — | 1.91 | — | 3.80 |

Minimal levels of Impurity A were observed in all formulations after 6 months storage at 2-8° C., 25° C./60% RH, and 40° C./75% RH. The formulations with increased levels of acid content showed decreased levels of Impurity B at 25° C./60% RH and 40° C./75% RH after 6 months storage. No growth of Impurity B was observed at 2-8° C. after 6 months storage.

1. A respirable dry powder, comprising respirable dry particles that comprise a tiotropium salt, one or more amino acids, acid content, sodium chloride, and optionally one or more additional therapeutic agents, wherein the tiotropium salt is about 0.01% to about 0.5%, the amino acid is about 5% to about 40%, the sodium chloride is about 50% to about 90%, the optional one or more additional therapeutic agents are up to about 30%, and the molar ratio of acid to amino acid is from about 0.002 to about 1, wherein all percentages are weight percentages on a dry basis and all the components of the respirable dry particles amount to 100%.

2. The respirable dry powder of claim 1, wherein when the respirable dry powder comprising respirable dry particles is sealed in a receptacle and stored for about 12 months at a temperature of about 15° C. to about 30° C., the purity of tiotropium is about 96.0% or greater.

3. The respirable dry powder of claim 1, wherein when the respirable dry powder comprising respirable dry particles is sealed in a receptacle and stored for about 12 months at a temperature of about 15° C. to about 30° C., the amount of tiotropium Impurity B is about 1.0% or less.

4. (canceled)

5. A respirable dry powder, comprising respirable dry particles that comprise a tiotropium salt, one or more amino acids, acid content, sodium chloride, and optionally one or more additional therapeutic agents, wherein the tiotropium salt is about 0.01% to about 0.5%, the amino acid is about 5% to about 40%, the sodium chloride is about 50% to about 90%, the optional one or more additional therapeutic agents are up to about 30%, and the molar ratio of acid to tiotropium is from about 2 to about 1000, wherein all percentages are weight percentages on a dry basis and all the components of the respirable dry particles amount to 100%.

6. A respirable dry powder, comprising respirable dry particles that comprise a tiotropium salt, one or more amino acids, acid content, sodium chloride, and optionally one or more additional therapeutic agents, wherein the tiotropium salt is about 0.01% to about 0.5%, the amino acid is about 5% to about 40%, the sodium chloride is about 50% to about 90%, the optional one or more additional therapeutic agents are up to about 30%, and the molar ratio of acid to tiotropium is from about 2 to about 1000, wherein all percentages are weight percentages on a dry basis and all the components of the respirable dry particles amount to 100%, and wherein when the respirable dry powder comprising respirable dry particles is sealed in a receptacle and stored for about 12 months at a temperature of about 15° C. to about 30° C., the purity of tiotropium is about 96.0% or greater.

7. The respirable dry powder of claim 1, wherein the one or more amino acids is leucine.

8. (canceled)

9. The respirable dry powder of claim 1, wherein the molar ratio of acid to amino acid is from about 0.005 to about 0.5.

10. (canceled)

11. The respirable dry powder of claim 5, wherein the molar ratio of acid to tiotropium is from about 5 to about 500.

12-15. (canceled)

16. The respirable dry powder of claim 1, wherein the tiotropium salt is about 0.02% to about 0.25%.

17. (canceled)

18. The respirable dry powder of claim 1, wherein the tiotropium salt is selected from the group consisting of tiotropium bromide, tiotropium chloride, and combinations thereof.

19-20. (canceled)

21. The respirable dry powder of claim 1, wherein the one or more additional therapeutic agents is present in an amount of about 0.01% to about 15%.

22. The respirable dry powder of claim 1, wherein the one or more additional therapeutic agents are independently selected from the group consisting of one or more inhaled corticosteroid, one or more long-acting beta agonist, one or more short-acting beta agonist, one or more bifunctional muscarinic antagonist-beta2 agonist, one or more anti-inflammatory agent, one or more bronchodilator, and any combination thereof.

23-26. (canceled)

27. The respirable dry powder of claim 1, wherein the amount of tiotropium Impurity A in the respirable dry powder in the sealed receptacle after about 12 months of storage at about 15° C. to about 30° C. is about 1.0% or less.

28. The respirable dry powder of claim 5, wherein the amount of tiotropium Impurity B in the respirable dry powder in the sealed receptacle after about 12 months of storage at about 15° C. to about 30° C. is about 1.0% or less.

29-33. (canceled)

34. The respirable dry powder of claim 1, wherein the respirable dry particles have a volume median geometric diameter (VMGD) of about 10 microns or less.

35. (canceled)

36. The respirable dry powder of claim 1, wherein the respirable dry particles have a tap density of greater than 0.4 g/cm3.

37-38. (canceled)

39. The respirable dry powder of claim 1, wherein the dry powder has a mass median aerodynamic diameter (MMAD) of between about 1 micron and about 5 microns.

40. The respirable dry powder of claim 1, wherein the respirable dry powder has a fine particle dose (FPD) less than 5 microns of about 1 microgram to about 5 micrograms of tiotropium.

41-45. (canceled)

46. The respirable dry powder of claim 1, wherein the dry particles have a 1/4 bar dispersibility ratio of about 1.5 or less as measured by laser diffraction.

47-111. (canceled)
